(19) United States
(12) Patent Application Publication
(10) Pub. No.: US 2026/0325516 A1
(43) Pub. Date: Oct. 22, 2026

(54) TREATING HIV WITH CABOTEGRAVIR AND RILPIVIRINE IN PEDIATRIC PATIENTS

(71) Applicants: ViiV Healthcare UK (No.3) Limited, Stevenage (GB); Janssen Sciences Ireland Unlimited Company, Co Cork (IE)

(72) Inventors: Sau Yan Amy CHEUNG, Brentford (GB); Herta Maria Ludovica CRAUWELS, Beerse (BE); Alberto RUSSU, Cologno (IT); Yu-Wei LIN, Victoria (AU)

(73) Assignees: ViiV Healthcare UK (No.3) Limited, Stevenage (GB); Janssen Sciences Ireland Unlimited Company, Co Cork (IE)

(21) Appl. No.: 19/108,818

(22) PCT Filed: Sep. 14, 2023
(86) PCT No.: PCT/EP2023/075264

§ 371 Date: Mar. 5, 2025

Related U.S. Application Data
(60) Provisional application No. 63/407,425, filed on Sep. 16, 2022.

(51) Int. Cl. A61K 31/505; A61K 31/4985; A61P 31/18

(52) U.S. Cl. CPC A61K 31/505; A61K 31/4985; A61P 31/18

(57) ABSTRACT

The disclosure provides a method of treating HIV infection in a human pediatric patient by the administration of cabotegravir or a pharmaceutically acceptable salt thereof, in combination with rilpivirine or a pharmaceutically acceptable salt thereof.

### FIELD

This disclosure relates to a method of treating HIV infection in a human pediatric patient by the administration of cabotegravir or a pharmaceutically acceptable salt thereof, in combination with rilpivirine or a pharmaceutically acceptable salt thereof.

### BACKGROUND

Decades of antiretroviral therapy (ART) advances have led to simplified treatment regimens for people living with human immunodeficiency virus (HIV-1). Viral suppression in adults is frequently achieved through close adherence to once daily, single-tablet ART regimens. However, the rates of viral suppression in children are lower when compared to those in adults. The lack of pediatric formulations and potent regimens in children contribute to difficulties in adherence and lower suppression rates. ART safety profiles have gradually improved, allowing for the inclusion of well-tolerated medications with infrequent adverse effects in treatment regimens. Despite ART advances for older populations, young children have access to fewer simplified HIV-1 regimens. The same hurdles that contribute to suboptimal pediatric ART adherence have also led to the deferment of pediatric clinical trials for medications already approved in adults, compounding the obstacles faced by children living with HIV-1.

Further, normal childhood weight gain requires periodic changes in weight-based ART dosing, making it difficult to produce a single fixed-dose combination formulation containing multiple antiretroviral medications whose doses can be adjusted as a child grows. In addition, young children also have difficulty swallowing tablets, requiring the development of specific drug formulations (i.e., liquids, dispersible tablets) aimed at this relatively small group of patients, further delaying access to new medications.

Cabotegravir (CAB) and rilpivirine (RPV) are the first long-acting (LA) injectable ARTs approved for the treatment of HIV-1. CAB is a potent integrase strand transfer inhibitor (INSTI) with attributes allowing formulation and delivery as a LA parenteral product. RPV, also formulated as a LA product, is a diaryl pyrimidine derivative and a potent non-nucleoside reverse transcriptase inhibitor (NNRTI). The combination of CAB LA plus RPV LA has an acceptable safety profile and is well-tolerated and efficacious as a dual injectable ART among virally suppressed adults and adolescents (over 12 years of age) living with HIV-1. CABENUVA is a 2-drug co-packaged product of cabotegravir and rilpivirine approved by the U.S. Food & Drug Administration for the treatment of HIV-1 infection in adults and adolescents 12 years of age and older and weighing at least 35 kg who are virologically suppressed (HIV-1 RNA <50 copies/mL) on a stable antiretroviral regimen with no history of treatment failure and with no known or suspected resistance to either cabotegravir or rilpivirine. The recommended dosing of CABENUVA is monthly or every two months.

### SUMMARY

In one aspect, the disclosure provides a method of treating HIV infection, the method comprising: administering to a human in need thereof a loading dose comprising (i) an intramuscular injection of cabotegravir or a pharmaceutically acceptable salt thereof and (ii) an intramuscular injection of rilpivirine or a pharmaceutically acceptable salt thereof; and thereafter administering one or more maintenance doses comprising (i) an intramuscular injection of cabotegravir or a pharmaceutically acceptable salt thereof and (ii) an intramuscular injection of rilpivirine or a pharmaceutically acceptable salt thereof; wherein the human is 2 years of age to less than 12 years of age.

In one aspect, the disclosure provides a method of treating HIV infection, the method comprising: administering to a human in need thereof a loading dose comprising (i) an intramuscular injection of cabotegravir or a pharmaceutically acceptable salt thereof and (ii) an intramuscular injection of rilpivirine or a pharmaceutically acceptable salt thereof; and thereafter administering one or more maintenance doses comprising (i) an intramuscular injection of cabotegravir or a pharmaceutically acceptable salt thereof and (ii) an intramuscular injection of rilpivirine or a pharmaceutically acceptable salt thereof; wherein the human is 2 years of age to less than 12 years of age; and wherein: (a) the human weighs 35 kg to less than 40 kg; the loading dose of cabotegravir or a pharmaceutically acceptable salt thereof is administered as a 600 mg intramuscular injection, and the loading dose of rilpivirine or a pharmaceutically acceptable salt thereof is administered as a 900 mg intramuscular injection; and the one or more maintenance doses of cabotegravir or a pharmaceutically acceptable salt thereof are administered as 400 mg intramuscular injections and the one or more maintenance doses of rilpivirine or a pharmaceutically acceptable salt thereof are administered as 600 mg intramuscular injections; (b) the human weighs 25 kg to 34.9 kg; the loading dose of cabotegravir or a pharmaceutically acceptable salt thereof is administered as a 300 mg intramuscular injection, and the loading dose of rilpivirine or a pharmaceutically acceptable salt thereof is administered as a 600 mg intramuscular injection; and the one or more maintenance doses of cabotegravir or a pharmaceutically acceptable salt thereof are administered as 200 mg intramuscular injections and the one or more maintenance doses of rilpivirine or a pharmaceutically acceptable salt thereof are administered as 450 mg intramuscular injections; (c) the human weighs 20 kg to 24.9 kg; the loading dose cabotegravir or a pharmaceutically acceptable salt thereof is administered as a 300 mg intramuscular injection and the loading dose of rilpivirine or a pharmaceutically acceptable salt thereof is administered as a 600 mg intramuscular injection; and the one or more maintenance doses of cabotegravir or a pharmaceutically acceptable salt thereof are administered as 200 mg intramuscular injections and the one or more maintenance doses of rilpivirine or a pharmaceutically acceptable salt thereof are administered as 450 mg intramuscular injections; (d) the human weighs 14 kg to 19.9 kg; the loading dose of cabotegravir or a pharmaceutically acceptable salt thereof is administered as a 300 mg intramuscular injection and the loading dose of rilpivirine or a pharmaceutically acceptable salt thereof is administered as a 450 mg intramuscular injection; and the one or more maintenance doses of cabotegravir or a pharmaceutically acceptable salt thereof are administered as 200 mg intramuscular injections and the one or more maintenance doses of rilpivirine or a pharmaceutically acceptable salt thereof are administered as 300 mg intramuscular injections; or (e) the human weighs 10 kg to 13.9 kg; the loading dose of cabotegravir or a pharmaceutically acceptable salt thereof is administered as a 300 mg intramuscular injection and the loading dose of rilpivirine or a pharmaceutically acceptable salt thereof is administered as a 450 mg intramuscular injection; and the one or more maintenance doses of cabotegravir or a pharmaceutically acceptable salt thereof are administered as 200 mg intramuscular injections and the one or more maintenance doses of rilpivirine or a pharmaceutically acceptable salt thereof are administered as 300 mg intramuscular injections.

In one aspect, the disclosure provides the use of (i) cabotegravir or a pharmaceutically acceptable salt thereof and (ii) rilpivirine or a pharmaceutically acceptable salt thereof in the manufacture of a medicament for use in the treatment of HIV infection in a human, wherein the human is 2 years of age to less than 12 years of age, and the medicament is formulated for regular intramuscular injections.

In one aspect, the disclosure provides cabotegravir or a pharmaceutically acceptable salt thereof and rilpivirine or a pharmaceutically acceptable salt thereof for use in treatment of HIV infection in a human, wherein the human is 2 years of age to less than 12 years of age, and the cabotegravir or a pharmaceutically acceptable salt thereof and rilpivirine or a pharmaceutically acceptable salt thereof are formulated for regular intramuscular injections.

In one aspect, the disclosure provides cabotegravir or a pharmaceutically acceptable salt thereof indicated in combination with rilpivirine or a pharmaceutically acceptable salt thereof for use in treatment of HIV infection in a human, wherein the human is 2 years of age to less than 12 years of age, and the cabotegravir or a pharmaceutically acceptable salt thereof is formulated for regular intramuscular injections. In an embodiment, the rilpivirine or a pharmaceutically acceptable salt thereof is formulated for intramuscular injection.

In one aspect, the disclosure provides rilpivirine or a pharmaceutically acceptable salt thereof indicated in combination with cabotegravir or a pharmaceutically acceptable salt thereof for use in treatment of HIV infection in a human, wherein the human is 2 years of age to less than 12 years of age, and the rilpivirine or a pharmaceutically acceptable salt thereof is formulated for regular intramuscular injections. In an embodiment, the cabotegravir or a pharmaceutically acceptable salt thereof is formulated for intramuscular injection.

The disclosed methods including the use of LA injectable ART advantageously improve treatment adherence rates compared to existing therapies, thereby allowing children living with HIV-1 to achieve sustained viral suppression.

### DETAILED DESCRIPTION

The present disclosure provides a method of treating HIV infection in a human pediatric patient by the administration of cabotegravir or a pharmaceutically acceptable salt thereof, in combination with rilpivirine or a pharmaceutically acceptable salt thereof. The method involves regularly administering intramuscular injections of (i) cabotegravir or a pharmaceutically acceptable salt thereof and (ii) rilpivirine or a pharmaceutically acceptable salt thereof to a human pediatric patient in need thereof. The human pediatric patient is 2 years of age to less than 12 years of age.

In some embodiments, the human is virally suppressed i.e., exhibits a viral load of less than or equal to, in particular, less than 50 copies of HIV-1 RNA per mL of blood plasma prior to beginning treatment. In some embodiments, the human exhibits a viral load of less than or equal to 50 copies of HIV-1 RNA per mL of blood plasma after at least 72 weeks of treatment with cabotegravir and rilpivirine, including an optional oral-lead in treatment.

### Intramuscular Dosing.

In one aspect, the disclosure provides a method of treating HIV infection, the method comprising: administering to a human in need thereof a loading dose comprising (i) an intramuscular injection of cabotegravir or a pharmaceutically acceptable salt thereof and (ii) an intramuscular injection of rilpivirine or a pharmaceutically acceptable salt thereof; and thereafter administering one or more maintenance doses comprising (i) an intramuscular injection of cabotegravir or a pharmaceutically acceptable salt thereof and (ii) an intramuscular injection of rilpivirine or a pharmaceutically acceptable salt thereof; wherein the human is 2 years of age to less than 12 years of age.

In some embodiments, the human weighs 10 kg to less than 40 kg, preferably 10 kg to 34.9 kg. In some embodiments, the human weighs 10 kg to 13.9 kg, 14 kg to 19.9 kg, 20 kg to 24.9 kg, 25 kg to 34.9 kg, or 35 kg to less than 40 kg. In some embodiments, the dose differs for patients in different weight bands, with patients in lighter weight bands receiving lower doses of cabotegravir or a pharmaceutically acceptable salt thereof and rilpivirine or a pharmaceutically acceptable salt thereof than patients in heavier weight bands.

In some embodiments, the loading dose of cabotegravir or a pharmaceutically acceptable salt thereof is greater than the one or more maintenance doses of cabotegravir or a pharmaceutically acceptable salt thereof. In some embodiments, the loading dose of rilpivirine or a pharmaceutically acceptable salt thereof is greater than the one or more maintenance doses of rilpivirine or a pharmaceutically acceptable salt thereof.

In some embodiments, the cabotegravir or a pharmaceutically acceptable salt thereof is administered as a 50 mg to 600 mg intramuscular injection, preferably a 100 mg to 600 mg intramuscular injection, a 150 mg to 600 mg intramuscular injection, a 200 mg to 600 mg intramuscular injection, a 300 mg to 600 mg intramuscular injection, or a 200 mg to 600 mg intramuscular injection. In an embodiment, the cabotegravir or a pharmaceutically acceptable salt thereof is administered as a 200 mg to 600 mg intramuscular injection. In an embodiment, the cabotegravir or a pharmaceutically acceptable salt thereof is administered as a 300 mg to 600 mg intramuscular injection. In an embodiment, the cabotegravir or a pharmaceutically acceptable salt thereof is administered as a 200 mg to 400 mg intramuscular injection. In some embodiments, the cabotegravir or a pharmaceutically acceptable salt thereof is administered as a 50 mg, 100 mg, 200 mg, 300 mg, 400 mg, 500 mg, or 600 mg intramuscular injection.

In some embodiments, the loading dose of cabotegravir or a pharmaceutically acceptable salt thereof is administered as a 50 mg to 600 mg intramuscular injection, preferably a 100 mg to 600 mg intramuscular injection, a 150 mg to 600 mg intramuscular injection, a 200 mg to 600 mg intramuscular injection, or a 300 mg to 600 mg intramuscular injection. In an embodiment, the loading dose of cabotegravir or a pharmaceutically acceptable salt thereof is administered as a 200 mg to 600 mg intramuscular injection. In an embodiment, the loading dose of cabotegravir or a pharmaceutically acceptable salt thereof is administered as a 300 mg to 600 mg intramuscular injection. In some embodiments, the loading dose of cabotegravir or a pharmaceutically acceptable salt thereof is administered as a 50 mg, 100 mg, 200 mg, 300 mg, 400 mg, 500 mg, or 600 mg intramuscular injection.

In some embodiments, the one or more maintenance doses of cabotegravir or a pharmaceutically acceptable salt thereof are administered as 50 mg to 600 mg intramuscular injections, preferably 100 mg to 600 mg intramuscular injections, 150 mg to 600 mg intramuscular injections, 200 mg to 600 mg intramuscular injections, or 200 mg to 400 mg intramuscular injections. In an embodiment, the one or more maintenance doses of cabotegravir or a pharmaceutically acceptable salt thereof are administered as 200 mg to 600 mg intramuscular injections. In an embodiment, the one or more maintenance doses of cabotegravir or a pharmaceutically acceptable salt thereof are administered as 200 mg to 400 mg intramuscular injections. In some embodiments, the one or more maintenance doses of cabotegravir or a pharmaceutically acceptable salt thereof are administered as 50 mg, 100 mg, 200 mg, 300 mg, 400 mg, 500 mg, or 600 mg intramuscular injections.

In some embodiments, the rilpivirine or a pharmaceutically acceptable salt thereof is administered as a 50 mg to 900 mg intramuscular injection, preferably a 100 mg to 900 mg intramuscular injection, a 150 mg to 900 mg intramuscular injection, a 200 mg to 900 mg intramuscular injection, a 250 mg to 900 mg intramuscular injection, a 300 mg to 900 mg intramuscular injection, a 450 mg to 900 mg intramuscular injection, or a 300 mg to 600 mg intramuscular injection. In an embodiment, the rilpivirine or a pharmaceutically acceptable salt thereof is administered as a 300 mg to 900 mg intramuscular injection. In an embodiment, the rilpivirine or a pharmaceutically acceptable salt thereof is administered as a 450 mg to 900 mg intramuscular injection. In an embodiment, the rilpivirine or a pharmaceutically acceptable salt thereof is administered as a 300 mg to 600 mg intramuscular injection. In some embodiments, the rilpivirine or a pharmaceutically acceptable salt thereof is administered as a 50 mg, 100 mg, 150 mg, 300 mg, 450 mg, 600 mg, 750 mg, or 900 mg intramuscular injection.

In some embodiments, the loading dose of rilpivirine or a pharmaceutically acceptable salt thereof is administered as a 50 mg to 900 mg intramuscular injection, preferably a 100 mg to 900 mg intramuscular injection, a 150 mg to 900 mg intramuscular injection, a 200 mg to 900 mg intramuscular injection, a 250 mg to 900 mg intramuscular injection, a 450 mg to 900 mg intramuscular injection, or a 300 mg to 900 mg intramuscular injection. In an embodiment, the loading dose of rilpivirine or a pharmaceutically acceptable salt thereof is administered as a 300 mg to 900 mg intramuscular injection. In an embodiment, the loading dose of rilpivirine or a pharmaceutically acceptable salt thereof is administered as a 450 mg to 900 mg intramuscular injection. In some embodiments, the loading dose of rilpivirine or a pharmaceutically acceptable salt thereof is administered as a 50 mg, 100 mg, 150 mg, 300 mg, 450 mg, 600 mg, 750 mg, or 900 mg intramuscular injection.

In some embodiments, the one or more maintenance doses of rilpivirine or a pharmaceutically acceptable salt thereof are administered as 50 mg to 900 mg intramuscular injections, preferably 100 mg to 900 mg intramuscular injections, 150 mg to 900 mg intramuscular injections, 200 mg to 900 mg intramuscular injections, 250 mg to 900 mg intramuscular injections, 300 mg to 900 mg intramuscular injections, or 300 mg to 600 mg intramuscular injections. In an embodiment, the maintenance dose of rilpivirine or a pharmaceutically acceptable salt thereof is administered as a 300 mg to 900 mg intramuscular injection. In an embodiment, the maintenance dose of rilpivirine or a pharmaceutically acceptable salt thereof is administered as a 300 mg to 600 mg intramuscular injection. In some embodiments, the one or more maintenance doses of rilpivirine or a pharmaceutically acceptable salt thereof are administered as 50 mg, 100 mg, 150 mg, 300 mg, 450 mg, 600 mg, 750 mg, or 900 mg intramuscular injections.

It should be understood that disclosure of an amount (e.g., in mg) of rilpivirine or a pharmaceutically acceptable salt thereof in the form of an injection includes the disclosed amount expressed as the base equivalent.

In some embodiments, the loading dose of cabotegravir or a pharmaceutically acceptable salt thereof and the loading dose of rilpivirine or a pharmaceutically acceptable salt thereof are administered less than 1 hour apart, preferably less than 30 minutes apart, less than 20 minutes apart, less than 15 minutes apart, less than 10 minutes apart, or less than 5 minutes apart. In some embodiments, for each maintenance dose of cabotegravir or a pharmaceutically acceptable salt thereof and rilpivirine or a pharmaceutically acceptable salt thereof, the cabotegravir or a pharmaceutically acceptable salt thereof and the rilpivirine or a pharmaceutically acceptable salt thereof are administered less than 1 hour apart, preferably less than 30 minutes apart, less than 20 minutes apart, less than 15 minutes apart, less than 10 minutes apart, or less than 5 minutes apart.

In some embodiments, the cabotegravir or a pharmaceutically acceptable salt thereof is administered first and the rilpivirine or a pharmaceutically acceptable salt thereof is administered second. In some embodiments, the rilpivirine or a pharmaceutically acceptable salt thereof is administered first and the cabotegravir or a pharmaceutically acceptable salt thereof is administered second.

In some embodiments, the intramuscular injections are administered at a site selected from a gluteus medius or a lateral aspect of a thigh.

In some embodiments, the intramuscular injections of cabotegravir or a pharmaceutically acceptable salt thereof are administered in a volume of 0.5 mL, 1 mL, 1.5 mL, 2 mL, 2.5 mL, or 3 mL. In some embodiments, the intramuscular injections of cabotegravir or a pharmaceutically acceptable salt thereof are at a concentration of 200 mg/mL and are administered in a volume of 0.5 mL, 1 mL, 1.5 mL, 2 mL, 2.5 mL or 3 mL. In some embodiments, the intramuscular injections of rilpivirine or a pharmaceutically acceptable salt thereof are administered in a volume of 0.5 mL, 1 mL, 1.5 mL, 2 mL, 2.5 mL or 3 mL. In some embodiments, the intramuscular injections of rilpivirine or a pharmaceutically acceptable salt thereof are at a concentration of 300 mg/mL and are administered in a volume of 0.5 mL, 1 mL, 1.5 mL, 2 mL, 2.5 mL or 3 mL.

### Intramuscular Dosing Interval.

In some embodiments, the one or more maintenance doses are administered at a regular interval. In some embodiments, the one or more maintenance doses are administered once every 4 weeks ±7 days or less frequently. In some embodiments, the one or more maintenance doses are administered once every month ±7 days or once every 4 weeks ±7 days. In some embodiments, the one or more maintenance doses are administered once every month ±7 days. In some embodiments, the one or more maintenance doses are administered once every 4 weeks ±7 days. In some embodiments, the one or more maintenance doses are administered once every two months ±7 days or once every 8 weeks ±7 days. In some embodiments, the one or more maintenance doses are administered once every two months ±7 days. In some embodiments, the one or more maintenance doses are administered every 8 weeks ±7 days.

In some embodiments, the one or more maintenance doses are administered at a regular interval for a period of time, and subsequently are administered at a different, e.g., longer, regular interval. In some embodiments, the one or more maintenance doses are administered once every month ±7 days or once every 4 weeks ±7 days, followed by once every two months ±7 days or once every 8 weeks ±7 days. In some embodiments, the one or more maintenance doses are administered once every month ±7 days or once every 4 weeks ±7 days for 1, 2, 3, 4, 5, 6, 7, 8, 9, 10, 11, 12, 13, 14, 15, 16, 17, 18, 19, 20, 21, 22, 23, or 24 times (i.e. 1, 2, 3, 4, 5, 6, 7, 8, 9, 10, 11, 12, 13, 14, 15, 16, 17, 18, 19, 20, 21, 22, 23, or 24 doses are given), followed by maintenance doses administered once every two months ±7 days or once every 8 weeks ±7 days. In some embodiments, the one or more maintenance doses are administered once every month ±7 days or once every 4 weeks ±7 days for 1, 2, 3, 4, 5, 6, 7, 8, 9, 10, 11, 12, 13, 14, 15, 16, 17, 18, 19, 20, 21, 22, 23, or 24 times, followed by maintenance doses administered once every two months ±7 days or once every 8 weeks ±7 days for at least for 1, 2, 3, 4, 5, 6, 7, 8, 9, 10, 11, 12, 13, 14, 15, 16, 17, 18, 19, 20, 21, 22, 23, or 24 times.

### Oral Lead-In Doses.

In some embodiments, oral lead-in doses of cabotegravir or a pharmaceutically acceptable salt thereof and rilpivirine or a pharmaceutically acceptable salt thereof are administered prior to the loading dose. Thus, in some embodiments, the method further comprises orally administering lead-in doses of cabotegravir or a pharmaceutically acceptable salt thereof and rilpivirine or a pharmaceutically acceptable salt thereof prior to the loading dose.

In some embodiments, the lead-in doses of cabotegravir or a pharmaceutically acceptable salt thereof are administered in the form of one or more tablets or one or more dispersible tablets. In some embodiments, the lead-in doses of rilpivirine or a pharmaceutically acceptable salt thereof are administered in the form of one or more tablets or one or more tablets dispersed in liquid.

In some embodiments, the lead-in doses of cabotegravir or a pharmaceutically acceptable salt thereof and rilpivirine or a pharmaceutically acceptable salt thereof are administered with food.

In some embodiments, the lead-in doses of cabotegravir or a pharmaceutically acceptable salt thereof are daily doses of 5 mg to 30 mg acid equivalent, preferably 10 mg to 30 mg acid equivalent. In some embodiments, the lead-in doses of cabotegravir or a pharmaceutically acceptable salt thereof are daily doses of 10 mg to 30 mg acid equivalent. In some embodiments, the lead-in doses of cabotegravir or a pharmaceutically acceptable salt thereof are daily doses of 5 mg, 10 mg, 15 mg, 20 mg, 25 mg, or 30 mg acid equivalent. In some embodiments, the lead-in doses of cabotegravir or a pharmaceutically acceptable salt thereof are daily doses of 10 mg acid equivalent. In some embodiments, the lead-in doses of cabotegravir or a pharmaceutically acceptable salt thereof are daily doses of 30 mg acid equivalent. In some embodiments, the lead-in doses of cabotegravir or a pharmaceutically acceptable salt thereof are administered as one 30 mg acid equivalent tablet. In some embodiments, the lead-in doses of cabotegravir or a pharmaceutically acceptable salt thereof are administered as one, two, three, four, or five 5 mg acid equivalent dispersible tablets. In some embodiments, the lead-in doses of cabotegravir or a pharmaceutically acceptable salt thereof are administered as two 5 mg acid equivalent dispersible tablets.

In some embodiments, the lead-in doses of rilpivirine or a pharmaceutically acceptable salt thereof are daily doses of 2.5 mg to 25 mg base equivalent, preferably 12.5 mg to 25 mg base equivalent. In some embodiments, the lead-in doses of rilpivirine or a pharmaceutically acceptable salt thereof are daily doses of 12.5 mg to 25 mg base equivalent. In some embodiments, the lead-in doses of rilpivirine or a pharmaceutically acceptable salt thereof are daily doses of 2.5 mg, 5 mg, 7.5 mg, 10 mg, 12.5 mg, 15 mg, 20 mg, or 25 mg base equivalent. In some embodiments, the lead-in doses of rilpivirine or a pharmaceutically acceptable salt thereof are daily doses of 12.5 mg base equivalent. In some embodiments, the lead-in doses of rilpivirine or a pharmaceutically acceptable salt thereof are daily doses of 15 mg base equivalent. In some embodiments, the lead-in doses of rilpivirine or a pharmaceutically acceptable salt thereof are daily doses of 25 mg base equivalent. In some embodiments, the lead-in doses of rilpivirine or a pharmaceutically acceptable salt thereof are administered as one 25 mg base equivalent tablet. In some embodiments, the lead-in doses of rilpivirine or a pharmaceutically acceptable salt thereof are administered as one, two, three, four, five, six, seven, or eight 2.5 mg tablets, preferably as tablets dispersed in liquid. In some embodiments, the lead-in doses of rilpivirine or a pharmaceutically acceptable salt thereof are administered as five 2.5 mg tablets, preferably as tablets dispersed in liquid. In some embodiments, the lead-in doses of rilpivirine or a pharmaceutically acceptable salt thereof are administered as six 2.5 mg tablets, preferably as tablets dispersed in liquid.

In some embodiments, the cabotegravir or a pharmaceutically acceptable salt thereof in the oral formulation is in the form of cabotegravir sodium. In some embodiments, the rilpivirine or a pharmaceutically acceptable salt thereof in the oral formulation is in the form of a pharmaceutically acceptable salt rilpivirine. In some embodiments, the pharmaceutically acceptable salt of rilpivirine is rilpivirine hydrochloride.

### Oral Bridging Doses.

In some embodiments, oral bridging doses of cabotegravir or a pharmaceutically acceptable salt thereof and rilpivirine or a pharmaceutically acceptable salt thereof are administered to a human in place of a scheduled intramuscular injection. Thus, in some embodiments, the method further comprises orally administering bridging doses, in particular once-daily bridging doses, of cabotegravir or a pharmaceutically acceptable salt thereof and/or rilpivirine or a pharmaceutically acceptable salt thereof in place of a scheduled intramuscular injection, particularly in a human who will miss or who has missed the scheduled intramuscular injection. In an embodiment, the first oral bridging dose is administered on, or ±7 days from, the date of the missed intramuscular injection. In an embodiment, the last oral bridging dose is administered on, in particular prior to, the date of resuming intramuscular injections.

### Formulations.

The injections of cabotegravir or a pharmaceutically acceptable salt thereof and rilpivirine or a pharmaceutically acceptable salt thereof are in the form of long-acting formulations.

In some embodiments, the long-acting formulation of cabotegravir or a pharmaceutically acceptable salt thereof is a suspension formulation, in particular a prolonged-release suspension for injection or an extended-release suspension for injection. In some embodiments, the cabotegravir or a pharmaceutically acceptable salt thereof in the long-acting formulation is in the form of the cabotegravir free base. In some embodiments, the concentration of cabotegravir or a pharmaceutically acceptable salt thereof in the suspension is 200 mg/mL. In some embodiments, the long-acting formulation of cabotegravir or a pharmaceutically acceptable salt thereof is present in a single-dose vial in an amount of 400 mg in a 2 mL volume. In some embodiments, the long-acting formulation of cabotegravir or a pharmaceutically acceptable salt thereof is present in a single-dose vial in an amount of 600 mg in a 3 mL volume. In some embodiments, the long-acting formulation of cabotegravir or a pharmaceutically acceptable salt thereof comprises cabotegravir or a pharmaceutically acceptable salt thereof, polysorbate, and polyethylene glycol. In some embodiments, the long-acting formulation of cabotegravir or a pharmaceutically acceptable salt thereof comprises cabotegravir or a pharmaceutically acceptable salt thereof, mannitol, polysorbate (e.g., polysorbate 20 or polysorbate 80), polyethylene glycol (PEG) (e.g., PEG 3350), and water for injection. In some embodiments, the long-acting formulation of cabotegravir or a pharmaceutically acceptable salt thereof comprises 200 mg/mL cabotegravir, 35 mg/mL mannitol, 20 mg/mL polysorbate 20, 20 mg/mL PEG 3350, and water for injection. In some embodiments, the long-acting formulation of cabotegravir or a pharmaceutically acceptable salt thereof comprises 200 mg/mL cabotegravir, 45 mg/mL mannitol, 20 mg/mL polysorbate 20, 20 mg/mL PEG 3350, and water for injection.

In some embodiments, the long-acting formulation of rilpivirine or a pharmaceutically acceptable salt thereof is a suspension formulation, in particular a prolonged-release suspension for injection or an extended-release suspension for injection. In some embodiments, the rilpivirine or a pharmaceutically acceptable salt thereof in the long-acting formulation is in the form of the rilpivirine free base. In some embodiments, the concentration of rilpivirine or a pharmaceutically acceptable salt thereof in the suspension is 300 mg/mL, in particular 300 mg base equivalent/mL. In some embodiments, the long-acting formulation of rilpivirine or a pharmaceutically acceptable salt thereof is present in a single-dose vial in an amount of 600 mg base equivalent in a 2 mL volume. In some embodiments, the long-acting formulation of rilpivirine or a pharmaceutically acceptable salt thereof is present in a single-dose vial in an amount of 900 mg base equivalent in a 3 mL volume. In some embodiments, the long-acting formulation of rilpivirine or a pharmaceutically acceptable salt thereof comprises rilpivirine or a pharmaceutically acceptable salt thereof and poloxamer. In some embodiments, the long-acting formulation of rilpivirine or a pharmaceutically acceptable salt thereof comprises rilpivirine or a pharmaceutically acceptable salt thereof, citric acid, and poloxamer. In some embodiments, the long-acting formulation of rilpivirine or a pharmaceutically acceptable salt thereof comprises 300 mg/mL rilpivirine, 1 mg/mL citric acid monohydrate, 50 mg/mL poloxamer 338, and water for injection. In some embodiments, the long-acting formulation of rilpivirine or a pharmaceutically acceptable salt thereof comprises 300 mg/mL rilpivirine, 1 mg/ml citric acid monohydrate, 50 mg/mL poloxamer 338, water for injection, glucose monohydrate, sodium dihydrogen phosphate monohydrate, and sodium hydroxide to adjust pH.

In some embodiments, the injections of cabotegravir or a pharmaceutically acceptable salt thereof are formulated as suspensions and the cabotegravir or a pharmaceutically acceptable salt thereof is in the free form (i.e., the non-salt form), and the injections of rilpivirine or a pharmaceutically acceptable salt thereof are formulated as suspensions and the rilpivirine or a pharmaceutically acceptable salt thereof is in the free form (i.e., the non-salt form). In some embodiments, the injections of cabotegravir or a pharmaceutically acceptable salt thereof are formulated as suspensions and the cabotegravir or a pharmaceutically acceptable salt thereof is in free acid/base form, and the injections of rilpivirine or a pharmaceutically acceptable salt thereof are formulated as suspensions and the rilpivirine or a pharmaceutically acceptable salt thereof is in free base form.

### Use in Treatment of HIV.

In one aspect, the disclosure provides the use of (i) cabotegravir or a pharmaceutically acceptable salt thereof and (ii) rilpivirine or a pharmaceutically acceptable salt thereof in the manufacture of a medicament for use in treatment of HIV infection in a human, wherein the human is 2 years of age to less than 12 years of age, and the medicament is formulated for regular intramuscular injections. In one aspect, the disclosure provides the use of a loading dose comprising (i) cabotegravir or a pharmaceutically acceptable salt thereof and (ii) rilpivirine or a pharmaceutically acceptable salt thereof; and one or more maintenance doses comprising (i) cabotegravir or a pharmaceutically acceptable salt thereof and (ii) rilpivirine or a pharmaceutically acceptable salt thereof in the manufacture of a medicament for use in treatment of HIV infection in a human, wherein the human is 2 years of age to less than 12 years of age, and the loading dose and one or more maintenance doses are formulated for intramuscular injection. In one aspect, the disclosure provides cabotegravir or a pharmaceutically acceptable salt thereof and rilpivirine or a pharmaceutically acceptable salt thereof for use in treatment of HIV infection in a human, wherein the human is 2 years of age to less than 12 years of age, and the cabotegravir or a pharmaceutically acceptable salt thereof and rilpivirine or a pharmaceutically acceptable salt thereof are formulated for regular intramuscular injections.

In some embodiments, the human weighs 10 kg to less than 40 kg, preferably 10 kg to 34.9 kg. In some embodiments, the human weighs 10 kg to 13.9 kg, 14 kg to 19.9 kg, 20 kg to 24.9 kg, 25 kg to 34.9 kg, or 35 kg to less than 40 kg. In some embodiments, the dose differs for use in patients in different weight bands, with patients in lighter weight bands using lower doses of cabotegravir or a pharmaceutically acceptable salt thereof and rilpivirine or a pharmaceutically acceptable salt thereof than patients in heavier weight bands.

In some embodiments, the loading dose of cabotegravir or a pharmaceutically acceptable salt thereof is greater than the one or more maintenance doses of cabotegravir or a pharmaceutically acceptable salt thereof. In some embodiments, the loading dose of rilpivirine or a pharmaceutically acceptable salt thereof is greater than the one or more maintenance doses of rilpivirine or a pharmaceutically acceptable salt thereof.

In some embodiments, the cabotegravir or a pharmaceutically acceptable salt thereof is formulated as a 50 mg to 600 mg intramuscular injection, preferably a 100 mg to 600 mg intramuscular injection, a 150 mg to 600 mg intramuscular injection, a 200 mg to 600 mg intramuscular injection, a 300 mg to 600 mg intramuscular injection, or a 200 mg to 400 mg intramuscular injection. In an embodiment, the cabotegravir or a pharmaceutically acceptable salt thereof is formulated as a 200 mg to 600 mg intramuscular injection. In an embodiment, the cabotegravir or a pharmaceutically acceptable salt thereof is formulated as a 300 mg to 600 mg intramuscular injection. In an embodiment, the cabotegravir or a pharmaceutically acceptable salt thereof is formulated as a 200 mg to 400 mg intramuscular injection. In some embodiments, the cabotegravir or a pharmaceutically acceptable salt thereof is formulated as a 50 mg, 100 mg, 200 mg, 300 mg, 400 mg, 500 mg, or 600 mg intramuscular injection.

In some embodiments, the loading dose of cabotegravir or a pharmaceutically acceptable salt thereof is formulated as a 50 mg to 600 mg intramuscular injection, preferably a 100 mg to 600 mg intramuscular injection, a 150 mg to 600 mg intramuscular injection, a 200 mg to 600 mg intramuscular injection, or a 300 mg to 600 mg intramuscular injection. In an embodiment, the loading dose of cabotegravir or a pharmaceutically acceptable salt thereof is formulated as a 200 mg to 600 mg intramuscular injection. In an embodiment, the loading dose of cabotegravir or a pharmaceutically acceptable salt thereof is formulated as a 300 mg to 600 mg intramuscular injection. In some embodiments, the loading dose of cabotegravir or a pharmaceutically acceptable salt thereof is formulated as a 50 mg, 100 mg, 200 mg, 300 mg, 400 mg, 500 mg, or 600 mg intramuscular injection.

In some embodiments, the one or more maintenance doses of cabotegravir or a pharmaceutically acceptable salt thereof are formulated as 50 mg to 600 mg intramuscular injections, preferably 100 mg to 600 mg intramuscular injections, 150 mg to 600 mg intramuscular injections, 200 mg to 600 mg intramuscular injections, or 200 mg to 400 mg intramuscular injections. In an embodiment, the one or more maintenance doses of cabotegravir or a pharmaceutically acceptable salt thereof are formulated as 200 mg to 600 mg intramuscular injections. In an embodiment, the one or more maintenance doses of cabotegravir or a pharmaceutically acceptable salt thereof are administered as 200 mg to 400 mg intramuscular injections. In some embodiments, the one or more maintenance doses of cabotegravir or a pharmaceutically acceptable salt thereof are formulated as 50 mg, 100 mg, 200 mg, 300 mg, 400 mg, 500 mg, or 600 mg intramuscular injections.

In some embodiments, the rilpivirine or a pharmaceutically acceptable salt thereof is formulated as a 50 mg to 900 mg intramuscular injection, preferably a 100 mg to 900 mg intramuscular injection, a 150 mg to 900 mg intramuscular injection, a 200 mg to 900 mg intramuscular injection, a 250 mg to 900 mg intramuscular injection, a 300 mg to 900 mg intramuscular injection, a 450 mg to 900 mg intramuscular injection, or a 300 mg to 600 mg intramuscular injection. In an embodiment, the rilpivirine or a pharmaceutically acceptable salt thereof is administered as a 300 mg to 900 mg intramuscular injection. In an embodiment, the rilpivirine or a pharmaceutically acceptable salt thereof is administered as a 450 mg to 900 mg intramuscular injection. In an embodiment, the rilpivirine or a pharmaceutically acceptable salt thereof is administered as a 300 mg to 600 mg intramuscular injection. In some embodiments, the rilpivirine or a pharmaceutically acceptable salt thereof is formulated as a 50 mg, 100 mg, 150 mg, 300 mg, 450 mg, 600 mg, 750 mg, or 900 mg intramuscular injection.

In some embodiments, the loading dose of rilpivirine or a pharmaceutically acceptable salt thereof is formulated as a 50 mg to 900 mg intramuscular injection, preferably a 100 mg to 900 mg intramuscular injection, a 150 mg to 900 mg intramuscular injection, a 200 mg to 900 mg intramuscular injection, a 250 mg to 900 mg intramuscular injection, a 450 mg to 900 mg intramuscular injection, or a 300 mg to 900 mg intramuscular injection. In an embodiment, the loading dose of rilpivirine or a pharmaceutically acceptable salt thereof is administered as a 300 mg to 900 mg intramuscular injection. In an embodiment, the loading dose of rilpivirine or a pharmaceutically acceptable salt thereof is administered as a 450 mg to 900 mg intramuscular injection. In some embodiments, the loading dose of rilpivirine or a pharmaceutically acceptable salt thereof is formulated as a 50 mg, 100 mg, 150 mg, 300 mg, 450 mg, 600 mg, 750 mg, or 900 mg intramuscular injection.

In some embodiments, the one or more maintenance doses of rilpivirine or a pharmaceutically acceptable salt thereof are formulated as 50 mg to 900 mg intramuscular injections, preferably 100 mg to 900 mg intramuscular injections, 150 mg to 900 mg intramuscular injections, 200 mg to 900 mg intramuscular injections, 250 mg to 900 mg intramuscular injections, 300 mg to 900 mg intramuscular injections, or 300 mg to 600 mg intramuscular injections. In an embodiment, the maintenance dose of rilpivirine or a pharmaceutically acceptable salt thereof is administered as a 300 mg to 900 mg intramuscular injection. In an embodiment, the maintenance dose of rilpivirine or a pharmaceutically acceptable salt thereof is administered as a 300 mg to 600 mg intramuscular injection. In some embodiments, the one or more maintenance doses of rilpivirine or a pharmaceutically acceptable salt thereof are formulated as 50 mg, 100 mg, 150 mg, 300 mg, 450 mg, 600 mg, 750 mg, or 900 mg intramuscular injections.

It should be understood that disclosure of an amount (e.g., in mg) of rilpivirine or a pharmaceutically acceptable salt thereof in the form of an injection includes the disclosed amount expressed as the base equivalent.

In some embodiments, the one or more maintenance doses are formulated for administration at a regular interval. In some embodiments, the one or more maintenance doses are formulated for administration once every 4 weeks #7 days or less frequently. In some embodiments, the one or more maintenance doses are formulated for administration once every month ±7 days or once every 4 weeks ±7 days. In some embodiments, the one or more maintenance doses are formulated for administration once every month ±7 days. In some embodiments, the one or more maintenance doses are formulated for administration once every 4 weeks ±7 days. In some embodiments, the one or more maintenance doses are formulated for administration once every two months ±7 days or once every 8 weeks ±7 days. In some embodiments, the one or more maintenance doses are formulated for administration once every two months ±7 days. In some embodiments, the one or more maintenance doses are formulated for administration every 8 weeks ±7 days.

In some embodiments, the one or more maintenance doses are formulated for administration at a regular interval for a period of time, followed by administration at a different, e.g., longer, regular interval. In some embodiments, the one or more maintenance doses are formulated for administration once every month ±7 days or once every 4 weeks ±7 days, followed by once every two months ±7 days or once every 8 weeks ±7 days. In some embodiments, the one or more maintenance doses are formulated for administration once every month ±7 days or once every 4 weeks ±7 days for 1, 2, 3, 4, 5, 6, 7, 8, 9, 10, 11, 12, 13, 14, 15, 16, 17, 18, 19, 20, 21, 22, 23, or 24 times (i.e. 1, 2, 3, 4, 5, 6, 7, 8, 9, 10, 11, 12, 13, 14, 15, 16, 17, 18, 19, 20, 21, 22, 23, or 24 doses are given), followed by once every two months ±7 days or once every 8 weeks ±7 days. In some embodiments, the one or more maintenance doses are formulated for administration once every month ±7 days or once every 4 weeks ±7 days for 1, 2, 3, 4, 5, 6, 7, 8, 9, 10, 11, 12, 13, 14, 15, 16, 17, 18, 19, 20, 21, 22, 23, or 24 times, followed by once every two months ±7 days or once every 8 weeks ±7 days for at least for 1, 2, 3, 4, 5, 6, 7, 8, 9, 10, 11, 12, 13, 14, 15, 16, 17, 18, 19, 20, 21, 22, 23, or 24 times.

In some embodiments, the medicament further comprises oral lead-in doses of cabotegravir or a pharmaceutically acceptable salt thereof and rilpivirine or a pharmaceutically acceptable salt thereof for administration prior to the loading dose. Thus, in some embodiments, the use further comprises lead-in doses of cabotegravir or a pharmaceutically acceptable salt thereof and rilpivirine or a pharmaceutically acceptable salt thereof formulated for oral administration prior to the loading dose.

In some embodiments, the lead-in doses of cabotegravir or a pharmaceutically acceptable salt thereof are formulated for administration in the form of one or more tablets or one or more dispersible tablets. In some embodiments, the lead-in doses of rilpivirine or a pharmaceutically acceptable salt thereof are formulated for administration in the form of one or more tablets or one or more tablets dispersed in liquid.

In some embodiments, the lead-in doses of cabotegravir or a pharmaceutically acceptable salt thereof and rilpivirine or a pharmaceutically acceptable salt thereof are formulated for administration with food.

In some embodiments, the lead-in doses of cabotegravir or a pharmaceutically acceptable salt thereof are daily doses of 5 mg to 30 mg acid equivalent, preferably 10 mg to 30 mg acid equivalent. In some embodiments, the lead-in doses of cabotegravir or a pharmaceutically acceptable salt thereof are daily doses of 10 mg to 30 mg acid equivalent. In some embodiments, the lead-in doses of cabotegravir or a pharmaceutically acceptable salt thereof are daily doses of 5 mg, 10 mg, 15 mg, 20 mg, 25 mg, or 30 mg acid equivalent. In some embodiments, the lead-in doses of cabotegravir or a pharmaceutically acceptable salt thereof are daily doses of 10 mg acid equivalent. In some embodiments, the lead-in doses of cabotegravir or a pharmaceutically acceptable salt thereof are daily doses of 30 mg acid equivalent. In some embodiments, the lead-in doses of cabotegravir or a pharmaceutically acceptable salt thereof are formulated for administration as one 30 mg acid equivalent tablet. In some embodiments, the lead-in doses of cabotegravir or a pharmaceutically acceptable salt thereof are formulated for administration as one, two, three, four, or five 5 mg acid equivalent dispersible tablets. In some embodiments, the lead-in doses of cabotegravir or a pharmaceutically acceptable salt thereof are formulated for administration as two 5 mg acid equivalent dispersible tablets.

In some embodiments, the lead-in doses of rilpivirine or a pharmaceutically acceptable salt thereof are daily doses of 2.5 mg to 25 mg base equivalent, preferably 12.5 mg to 25 mg base equivalent. In some embodiments, the lead-in doses of rilpivirine or a pharmaceutically acceptable salt thereof are daily doses of 12.5 mg to 25 mg base equivalent. In some embodiments, the lead-in doses of rilpivirine or a pharmaceutically acceptable salt thereof are daily doses of 2.5 mg, 5 mg, 7.5 mg, 10 mg, 12.5 mg, 15 mg, 20 mg, or 25 mg base equivalent. In some embodiments, the lead-in doses of rilpivirine or a pharmaceutically acceptable salt thereof are daily doses of 12.5 mg base equivalent. In some embodiments, the lead-in doses of rilpivirine or a pharmaceutically acceptable salt thereof are daily doses of 15 mg base equivalent. In some embodiments, the lead-in doses of rilpivirine or a pharmaceutically acceptable salt thereof are daily doses of 25 mg base equivalent. In some embodiments, the lead-in doses of rilpivirine or a pharmaceutically acceptable salt thereof are formulated for administration as one 25 mg base equivalent tablet. In some embodiments, the lead-in doses of rilpivirine or a pharmaceutically acceptable salt thereof are formulated for administration as one, two, three, four, five, six, seven, or eight 2.5 mg tablets, preferably as tablets dispersed in liquid. In some embodiments, the lead-in doses of rilpivirine or a pharmaceutically acceptable salt thereof are formulated for administration as five 2.5 mg tablets, preferably as tablets dispersed in liquid. In some embodiments, the lead-in doses of rilpivirine or a pharmaceutically acceptable salt thereof are formulated for administration as six 2.5 mg tablets, preferably as tablets dispersed in liquid.

In some embodiments, the cabotegravir or a pharmaceutically acceptable salt thereof in the oral formulation is in the form of cabotegravir sodium. In some embodiments, the rilpivirine or a pharmaceutically acceptable salt thereof in the oral formulation is in the form of a pharmaceutically acceptable salt rilpivirine. In some embodiments, the pharmaceutically acceptable salt of rilpivirine is rilpivirine hydrochloride.

In some embodiments, the medicament comprises oral bridging doses of cabotegravir or a pharmaceutically acceptable salt thereof and rilpivirine or a pharmaceutically acceptable salt thereof for administration to a human in place of a scheduled intramuscular injection. Thus, in some embodiments, the use further comprises oral bridging doses, in particular once-daily oral bridging doses, of cabotegravir or a pharmaceutically acceptable salt thereof and/or rilpivirine or a pharmaceutically acceptable salt thereof for administration in place of a scheduled intramuscular injection, particularly for a human who will miss or who has missed the scheduled intramuscular injection. In an embodiment, the first oral bridging dose is provided on, or ±7 days from, the date of the missed intramuscular injection. In an embodiment, the last oral bridging dose is provided on, in particular prior to, the date of resuming intramuscular injections.

In some embodiments, the medicament is suitable for administration to a human exhibiting a viral load of less than or equal to 50 copies of HIV-1 RNA per mL of blood plasma. In some embodiments, use of the medicament in the human achieves a viral load of less than or equal to 50 copies of HIV-1 RNA per mL of blood plasma after at least 72 weeks.

In some embodiments, the loading dose of cabotegravir or a pharmaceutically acceptable salt thereof and the loading dose of rilpivirine or a pharmaceutically acceptable salt thereof are formulated for administration less than 1 hour apart, preferably less than 30 minutes apart, less than 20 minutes apart, less than 15 minutes apart, less than 10 minutes apart, or less than 5 minutes apart. In some embodiments, for each maintenance dose of cabotegravir or a pharmaceutically acceptable salt thereof and rilpivirine or a pharmaceutically acceptable salt thereof, the cabotegravir or a pharmaceutically acceptable salt thereof and the rilpivirine or a pharmaceutically acceptable salt thereof are formulated for administration less than 1 hour apart, preferably less than 30 minutes apart, less than 20 minutes apart, less than 15 minutes apart, less than 10 minutes apart, or less than 5 minutes apart.

In some embodiments, the cabotegravir or a pharmaceutically acceptable salt thereof is formulated for administration first and the rilpivirine or a pharmaceutically acceptable salt thereof is formulated for administration second. In some embodiments, the rilpivirine or a pharmaceutically acceptable salt thereof is formulated for administration first and the cabotegravir or a pharmaceutically acceptable salt thereof is formulated for administration second.

In some embodiments, the intramuscular injections are formulated for administration at a site selected from a gluteus medius or a lateral aspect of a thigh.

In some embodiments, the intramuscular injections of cabotegravir or a pharmaceutically acceptable salt thereof are formulated in a volume of 0.5 mL, 1 mL, 1.5 mL, 2 mL, 2.5 mL, or 3 mL. In some embodiments, the intramuscular injections of cabotegravir or a pharmaceutically acceptable salt thereof are formulated at a concentration of 200 mg/mL and in a volume of 0.5 mL, 1 mL, 1.5 mL, 2 mL, 2.5 mL or 3 mL. In some embodiments, the intramuscular injections of rilpivirine or a pharmaceutically acceptable salt thereof are formulated in a volume of 0.5 mL, 1 mL, 1.5 mL, 2 mL, 2.5 mL or 3 mL. In some embodiments, the intramuscular injections of rilpivirine or a pharmaceutically acceptable salt thereof are formulated at a concentration of 300 mg/mL and in a volume of 0.5 mL, 1 mL, 1.5 mL, 2 mL, 2.5 mL or 3 mL.

The injections of cabotegravir or a pharmaceutically acceptable salt thereof and rilpivirine or a pharmaceutically acceptable salt thereof are in the form of long-acting formulations.

In some embodiments, the long-acting formulation of cabotegravir or a pharmaceutically acceptable salt thereof is a suspension formulation, in particular a prolonged-release suspension for injection or an extended-release suspension for injection. In some embodiments, the concentration of cabotegravir or a pharmaceutically acceptable salt thereof in the suspension is 200 mg/mL. In some embodiments, the long-acting formulation of cabotegravir or a pharmaceutically acceptable salt thereof is present in a single-dose vial in an amount of 400 mg in a 2 mL volume. In some embodiments, the long-acting formulation of cabotegravir or a pharmaceutically acceptable salt thereof is present in a single-dose vial in an amount of 600 mg in a 3 mL volume. In some embodiments, the long-acting formulation of cabotegravir or a pharmaceutically acceptable salt thereof comprises cabotegravir or a pharmaceutically acceptable salt thereof, polysorbate, and polyethylene glycol. In some embodiments, the long-acting formulation of cabotegravir or a pharmaceutically acceptable salt thereof comprises cabotegravir or a pharmaceutically acceptable salt thereof, mannitol, polysorbate (e.g., polysorbate 20 or polysorbate 80), polyethylene glycol (PEG) (e.g., PEG 3350), and water for injection. In some embodiments, the long-acting formulation of cabotegravir or a pharmaceutically acceptable salt thereof comprises 200 mg/mL cabotegravir, 35 mg/mL mannitol, 20 mg/mL polysorbate 20, 20 mg/mL PEG 3350, and water for injection. In some embodiments, the long-acting formulation of cabotegravir or a pharmaceutically acceptable salt thereof comprises 200 mg/mL cabotegravir, 45 mg/mL mannitol, 20 mg/mL polysorbate 20, 20 mg/mL PEG 3350, and water for injection. In some embodiments, the cabotegravir or a pharmaceutically acceptable salt thereof in the long-acting formulation is in the form of the cabotegravir free base.

In some embodiments, the long-acting formulation of rilpivirine or a pharmaceutically acceptable salt thereof is a suspension formulation, in particular a prolonged-release suspension for injection or an extended-release suspension for injection. In some embodiments, the concentration of rilpivirine or a pharmaceutically acceptable salt thereof in the suspension is 300 mg/mL, in particular 300 mg base equivalent/mL. In some embodiments, the long-acting formulation of rilpivirine or a pharmaceutically acceptable salt thereof is present in a single-dose vial in an amount of 600 mg base equivalent in a 2 mL volume. In some embodiments, the long-acting formulation of rilpivirine or a pharmaceutically acceptable salt thereof is present in a single-dose vial in an amount of 900 mg base equivalent in a 3 mL volume. In some embodiments, the long-acting formulation of rilpivirine or a pharmaceutically acceptable salt thereof comprises rilpivirine or a pharmaceutically acceptable salt thereof and poloxamer. In some embodiments, the long-acting formulation of rilpivirine or a pharmaceutically acceptable salt thereof comprises rilpivirine or a pharmaceutically acceptable salt thereof, citric acid, and poloxamer. In some embodiments, the long-acting formulation of rilpivirine or a pharmaceutically acceptable salt thereof comprises 300 mg/mL rilpivirine, 1 mg/mL citric acid monohydrate, 50 mg/mL poloxamer 338, and water for injection. In some embodiments, the long-acting formulation of rilpivirine or a pharmaceutically acceptable salt thereof comprises 300 mg/mL rilpivirine, 1 mg/mL citric acid monohydrate, 50 mg/mL poloxamer 338, water for injection, glucose monohydrate, sodium dihydrogen phosphate monohydrate, and sodium hydroxide to adjust pH. In some embodiments, the rilpivirine or a pharmaceutically acceptable salt thereof in the long-acting formulation is in the form of the rilpivirine free base.

In some embodiments, the injections of cabotegravir or a pharmaceutically acceptable salt thereof are formulated as suspensions and the cabotegravir or a pharmaceutically acceptable salt thereof is in the free form (i.e., the non-salt form), and the injections of rilpivirine or a pharmaceutically acceptable salt thereof are formulated as suspensions and the rilpivirine or a pharmaceutically acceptable salt thereof is in the free form (i.e., the non-salt form). In some embodiments, the injections of cabotegravir or a pharmaceutically acceptable salt thereof are formulated as suspensions and the cabotegravir or a pharmaceutically acceptable salt thereof is in free acid/base form, and the injections of rilpivirine or a pharmaceutically acceptable salt thereof are formulated as suspensions and the rilpivirine or a pharmaceutically acceptable salt thereof is in free base form.

### Compositions of Cabotegravir and Rilpivirine.

In one aspect, the disclosure provides cabotegravir or a pharmaceutically acceptable salt thereof indicated in combination with rilpivirine or a pharmaceutically acceptable salt thereof for use in treatment of HIV infection in a human, wherein the human is 2 years of age to less than 12 years of age, and the cabotegravir or a pharmaceutically acceptable salt thereof is formulated for regular intramuscular injections. In an embodiment, the rilpivirine or a pharmaceutically acceptable salt thereof is formulated for regular intramuscular injections. In one aspect, the disclosure provides rilpivirine or a pharmaceutically acceptable salt thereof indicated in combination with cabotegravir or a pharmaceutically acceptable salt thereof for use in treatment of HIV infection in a human, wherein the human is 2 years of age to less than 12 years of age, and the rilpivirine or a pharmaceutically acceptable salt thereof is formulated for regular intramuscular injections. In an embodiment, the cabotegravir or a pharmaceutically aceotable salt thereof is formulated for regular intramuscular injections.

In some embodiments, the human weighs 10 kg to less than 40 kg, preferably 10 kg to 34.9 kg. In some embodiments, the human weighs 10 kg to 13.9 kg, 14 kg to 19.9 kg, 20 kg to 24.9 kg, 25 kg to 34.9 kg, or 35 kg to less than 40 kg. In some embodiments, the dose differs for patients in different weight bands, with lower doses of cabotegravir or a pharmaceutically acceptable salt thereof and rilpivirine or a pharmaceutically acceptable salt thereof being suitable for patients in lighter weight bands compared to patients in heavier weight bands.

In some embodiments, the cabotegravir or a pharmaceutically acceptable salt thereof is provided as a loading dose and as one or more maintenance doses, and the loading dose is greater than the one or more maintenance doses. In some embodiments, the rilpivirine or a pharmaceutically acceptable salt thereof is provided as a loading dose and as one or more maintenance doses, and the loading dose is greater than the one or more maintenance doses.

In some embodiments, the cabotegravir or a pharmaceutically acceptable salt thereof is provided as a 50 mg to 600 mg dose suitable for intramuscular injection, preferably a 100 mg to 600 mg dose, a 150 mg to 600 mg dose, a 200 mg to 600 mg dose, a 300 mg to 600 mg dose, or a 200 mg to 40 mg dose. In an embodiment, the cabotegravir or a pharmaceutically acceptable salt thereof is provided as a 200 mg to 600 mg dose suitable for intramuscular injection. In an embodiment, the cabotegravir or a pharmaceutically acceptable salt thereof is provided as a 300 mg to 600 mg dose suitable for intramuscular injection. In an embodiment, the cabotegravir or a pharmaceutically acceptable salt thereof is provided as a 200 mg to 400 mg dose suitable for intramuscular injection. In some embodiments, the cabotegravir or a pharmaceutically acceptable salt thereof is provided as a 50 mg, 100 mg, 200 mg, 300 mg, 400 mg, 500 mg, or 600 mg dose suitable for intramuscular injection.

In some embodiments, the loading dose of cabotegravir or a pharmaceutically acceptable salt thereof is provided as a 50 mg to 600 mg dose suitable for intramuscular injection, preferably a 100 mg to 600 mg dose suitable for intramuscular injection, a 150 mg to 600 mg dose suitable for intramuscular injection, a 200 mg to 600 mg dose suitable for intramuscular injection, or a 300 mg to 600 mg dose suitable for intramuscular injection. In an embodiment, the loading dose of cabotegravir or a pharmaceutically acceptable salt thereof is provided as a 200 mg to 600 mg dose suitable for intramuscular injection. In an embodiment, the loading dose of cabotegravir or a pharmaceutically acceptable salt thereof is provided as a 300 mg to 600 mg dose suitable for intramuscular injection. In some embodiments, the loading dose of cabotegravir or a pharmaceutically acceptable salt thereof is provided as a 50 mg, 100 mg, 200 mg, 300 mg, 400 mg, 500 mg, or 600 mg dose suitable for intramuscular injection.

In some embodiments, the one or more maintenance doses of cabotegravir or a pharmaceutically acceptable salt thereof are provided as 50 mg to 600 mg doses suitable for intramuscular injections, preferably 100 mg to 600 mg doses suitable for intramuscular injections, 150 mg to 600 mg doses suitable for intramuscular injections, 200 mg to 600 mg doses suitable for intramuscular injections, or 200 mg to 400 mg doses suitable for intramuscular injections. In an embodiment, the one or more maintenance doses of cabotegravir or a pharmaceutically acceptable salt thereof are provided as 200 mg to 600 mg doses suitable for intramuscular injections. In an embodiment, the one or more maintenance doses of cabotegravir or a pharmaceutically acceptable salt thereof are provided as 200 mg to 400 mg doses suitable for intramuscular injections. In some embodiments, the one or more maintenance doses of cabotegravir or a pharmaceutically acceptable salt thereof are provided as 50 mg, 100 mg, 200 mg, 300 mg, 400 mg, 500 mg, or 600 mg doses suitable for intramuscular injections.

In some embodiments, the rilpivirine or a pharmaceutically acceptable salt thereof is provided as a 50 mg to 900 mg dose suitable for intramuscular injection, preferably a 100 mg to 900 mg dose suitable for intramuscular injection, a 150 mg to 900 mg dose suitable for intramuscular injection, a 200 mg to 900 mg dose suitable for intramuscular injection, a 250 mg to 900 mg dose suitable for intramuscular injection, a 300 mg to 900 mg dose suitable for intramuscular injection, a 450 mg to 900 mg dose suitable for intramuscular injection, or a 300 mg to 600 mg dose suitable for intramuscular injection. In an embodiment, the rilpivirine or a pharmaceutically acceptable salt thereof is administered as a 300 mg to 900 mg intramuscular injection. In an embodiment, the rilpivirine or a pharmaceutically acceptable salt thereof is administered as a 450 mg to 900 mg intramuscular injection. In an embodiment, the rilpivirine or a pharmaceutically acceptable salt thereof is administered as a 300 mg to 600 mg intramuscular injection. In some embodiments, the rilpivirine or a pharmaceutically acceptable salt thereof is provided as a 50 mg, 100 mg, 150 mg, 300 mg, 450 mg, 600 mg, 750 mg, or 900 mg dose suitable for intramuscular injection.

In some embodiments, the loading dose of rilpivirine or a pharmaceutically acceptable salt thereof is provided as a 50 mg to 900 mg dose suitable for intramuscular injection, preferably a 100 mg to 900 mg dose suitable for intramuscular injection, a 150 mg to 900 mg dose suitable for intramuscular injection, a 200 mg to 900 mg dose suitable for intramuscular injection, a 250 mg to 900 mg dose suitable for intramuscular injection, a 300 mg to 900 mg dose suitable for intramuscular injection, or a 450 mg to 900 mg dose suitable for intramuscular injection. In an embodiment, the loading dose of rilpivirine or a pharmaceutically acceptable salt thereof is administered as a 300 mg to 900 mg intramuscular injection. In an embodiment, the loading dose of rilpivirine or a pharmaceutically acceptable salt thereof is administered as a 450 mg to 900 mg intramuscular injection. In some embodiments, the loading dose of rilpivirine or a pharmaceutically acceptable salt thereof is provided as a 50 mg, 100 mg, 150 mg, 300 mg, 450 mg, 600 mg, 750 mg, or 900 mg dose suitable for intramuscular injection.

In some embodiments, the one or more maintenance doses of rilpivirine or a pharmaceutically acceptable salt thereof are provided as 50 mg to 900 mg doses suitable for intramuscular injections, preferably 100 mg to 900 mg doses suitable for intramuscular injections, 150 mg to 900 mg doses suitable for intramuscular injections, 200 mg to 900 mg doses suitable for intramuscular injections, 250 mg to 900 mg doses suitable for intramuscular injections, 300 mg to 900 mg doses suitable for intramuscular injections, or 300 mg to 600 mg doses suitable for intramuscular injections. In an embodiment, the maintenance dose of rilpivirine or a pharmaceutically acceptable salt thereof is administered as a 300 mg to 900 mg intramuscular injection. In an embodiment, the maintenance dose of rilpivirine or a pharmaceutically acceptable salt thereof is administered as a 300 mg to 600 mg intramuscular injection. In some embodiments, the one or more maintenance doses of rilpivirine or a pharmaceutically acceptable salt thereof are provided as 50 mg, 100 mg, 150 mg, 300 mg, 450 mg, 600 mg, 750 mg, or 900 mg doses suitable for intramuscular injections.

It should be understood that disclosure of an amount (e.g., in mg) of rilpivirine or a pharmaceutically acceptable salt thereof in the form of an injection includes the disclosed amount expressed as the base equivalent.

In some embodiments, the cabotegravir or a pharmaceutically acceptable salt thereof and rilpivirine or a pharmaceutically acceptable salt further comprise oral lead-in doses suitable for administration prior to the loading dose. Thus, in some embodiments, the cabotegravir or a pharmaceutically acceptable salt thereof and rilpivirine or a pharmaceutically acceptable salt thereof formulated further comprise lead-in doses suitable for oral administration prior to the loading dose.

In some embodiments, the lead-in doses of cabotegravir or a pharmaceutically acceptable salt thereof are provided for administration in the form of one or more tablets or one or more dispersible tablets. In some embodiments, the lead-in doses of rilpivirine or a pharmaceutically acceptable salt thereof are provided for administration in the form of one or more tablets or one or more tablets dispersed in liquid.

In some embodiments, the lead-in doses of cabotegravir or a pharmaceutically acceptable salt thereof and rilpivirine or a pharmaceutically acceptable salt thereof are suitable for administration with food.

In some embodiments, the lead-in doses of cabotegravir or a pharmaceutically acceptable salt thereof are daily doses of 5 mg to 30 mg acid equivalent, preferably 10 mg to 30 mg acid equivalent. In some embodiments, the lead-in doses of cabotegravir or a pharmaceutically acceptable salt thereof are daily doses of 10 mg to 30 mg acid equivalent. In some embodiments, the lead-in doses of cabotegravir or a pharmaceutically acceptable salt thereof are daily doses of 5 mg, 10 mg, 15 mg, 20 mg, 25 mg, or 30 mg acid equivalent. In some embodiments, the lead-in doses of cabotegravir or a pharmaceutically acceptable salt thereof are daily doses of 10 mg acid equivalent. In some embodiments, the lead-in doses of cabotegravir or a pharmaceutically acceptable salt thereof are daily doses of 30 mg acid equivalent. In some embodiments, the lead-in doses of cabotegravir or a pharmaceutically acceptable salt thereof are provided for administration as one 30 mg acid equivalent tablet. In some embodiments, the lead-in doses of cabotegravir or a pharmaceutically acceptable salt thereof are provided for administration as one, two, three, four, or five 5 mg acid equivalent dispersible tablets. In some embodiments, the lead-in doses of cabotegravir or a pharmaceutically acceptable salt thereof are provided for administration as two 5 mg acid equivalent dispersible tablets.

In some embodiments, the lead-in doses of rilpivirine or a pharmaceutically acceptable salt thereof are daily doses of 2.5 mg to 25 mg base equivalent, preferably 12.5 mg to 25 mg base equivalent. In some embodiments, the lead-in doses of rilpivirine or a pharmaceutically acceptable salt thereof are daily doses of 12.5 mg to 25 mg base equivalent. In some embodiments, the lead-in doses of rilpivirine or a pharmaceutically acceptable salt thereof are daily doses of 2.5 mg, 5 mg, 7.5 mg, 10 mg, 12.5 mg, 15 mg, 20 mg, or 25 mg base equivalent. In some embodiments, the lead-in doses of rilpivirine or a pharmaceutically acceptable salt thereof are daily doses of 12.5 mg base equivalent. In some embodiments, the lead-in doses of rilpivirine or a pharmaceutically acceptable salt thereof are daily doses of 15 mg base equivalent. In some embodiments, the lead-in doses of rilpivirine or a pharmaceutically acceptable salt thereof are daily doses of 25 mg base equivalent. In some embodiments, the lead-in doses of rilpivirine or a pharmaceutically acceptable salt thereof are provided for administration as one 25 mg base equivalent tablet. In some embodiments, the lead-in doses of rilpivirine or a pharmaceutically acceptable salt thereof are provided for administration as one, two, three, four, five, six, seven, or eight 2.5 mg tablets, preferably as tablets dispersed in liquid. In some embodiments, the lead-in doses of rilpivirine or a pharmaceutically acceptable salt thereof are formulated for administration as five 2.5 mg tablets, preferably as tablets dispersed in liquid. In some embodiments, the lead-in doses of rilpivirine or a pharmaceutically acceptable salt thereof are formulated for administration as six 2.5 mg tablets, preferably as tablets dispersed in liquid.

In some embodiments, the cabotegravir or a pharmaceutically acceptable salt thereof in the oral formulation is in the form of cabotegravir sodium. In some embodiments, the rilpivirine or a pharmaceutically acceptable salt thereof in the oral formulation is in the form of a pharmaceutically acceptable salt rilpivirine. In some embodiments, the pharmaceutically acceptable salt of rilpivirine is rilpivirine hydrochloride.

In some embodiments, oral bridging doses of cabotegravir or a pharmaceutically acceptable salt thereof and rilpivirine or a pharmaceutically acceptable salt thereof are provided to a human in place of a scheduled intramuscular injection. Thus, in some embodiments, the use further comprises providing oral bridging doses, in particular once-daily bridging doses, of cabotegravir or a pharmaceutically acceptable salt thereof and/or rilpivirine or a pharmaceutically acceptable salt thereof in place of a scheduled intramuscular injection, particularly for a human who will miss or who has missed the scheduled intramuscular injection. In an embodiment, the first oral bridging dose is provided on, or ±7 days from, the date of the missed intramuscular injection. In an embodiment, the last oral bridging dose is provided on, in particular prior to, the date of resuming intramuscular injections.

In some embodiments, the cabotegravir or a pharmaceutically acceptable salt thereof and rilpivirine or a pharmaceutically acceptable salt thereof are suitable for administration to a human exhibiting a viral load of less than or equal to 50 copies of HIV-1 RNA per mL of blood plasma. In some embodiments, the cabotegravir or a pharmaceutically acceptable salt thereof and rilpivirine or a pharmaceutically acceptable salt thereof achieve a viral load in the human of less than or equal to 50 copies of HIV-1 RNA per mL of blood plasma after at least 72 weeks.

The injections of cabotegravir or a pharmaceutically acceptable salt thereof and rilpivirine or a pharmaceutically acceptable salt thereof are in the form of long-acting formulations.

In some embodiments, the long-acting formulation of cabotegravir or a pharmaceutically acceptable salt thereof is a suspension formulation, in particular a prolonged-release suspension for injection or an extended-release suspension for injection. In some embodiments, the concentration of cabotegravir or a pharmaceutically acceptable salt thereof in the suspension is 200 mg/mL. In some embodiments, the long-acting formulation of cabotegravir or a pharmaceutically acceptable salt thereof is present in a single-dose vial in an amount of 400 mg in a 2 mL volume. In some embodiments, the long-acting formulation of cabotegravir or a pharmaceutically acceptable salt thereof is present in a single-dose vial in an amount of 600 mg in a 3 mL volume. In some embodiments, the long-acting formulation of cabotegravir or a pharmaceutically acceptable salt thereof comprises cabotegravir or a pharmaceutically acceptable salt thereof, polysorbate, and polyethylene glycol. In some embodiments, the long-acting formulation of cabotegravir or a pharmaceutically acceptable salt thereof comprises cabotegravir or a pharmaceutically acceptable salt thereof, mannitol, polysorbate (e.g., polysorbate 20 or polysorbate 80), polyethylene glycol (PEG) (e.g., PEG 3350), and water for injection. In some embodiments, the long-acting formulation of cabotegravir or a pharmaceutically acceptable salt thereof comprises 200 mg/mL cabotegravir, 35 mg/mL mannitol, 20 mg/mL polysorbate 20, 20 mg/mL PEG 3350, and water for injection. In some embodiments, the long-acting formulation of cabotegravir or a pharmaceutically acceptable salt thereof comprises 200 mg/mL cabotegravir, 45 mg/mL mannitol, 20 mg/mL polysorbate 20, 20 mg/mL PEG 3350, and water for injection. In some embodiments, the cabotegravir or a pharmaceutically acceptable salt thereof in the long-acting formulation is in the form of the cabotegravir free base.

In some embodiments, the long-acting formulation of rilpivirine or a pharmaceutically acceptable salt thereof is a suspension formulation, in particular a prolonged-release suspension for injection or an extended-release suspension for injection. In some embodiments, the concentration of rilpivirine or a pharmaceutically acceptable salt thereof in the suspension is 300 mg/mL, in particular 300 mg base equivalent/mL. In some embodiments, the long-acting formulation of rilpivirine or a pharmaceutically acceptable salt thereof is present in a single-dose vial in an amount of 600 mg base equivalent in a 2 mL volume. In some embodiments, the long-acting formulation of rilpivirine or a pharmaceutically acceptable salt thereof is present in a single-dose vial in an amount of 900 mg base equivalent in a 3 mL volume. In some embodiments, the long-acting formulation of rilpivirine or a pharmaceutically acceptable salt thereof comprises rilpivirine or a pharmaceutically acceptable salt thereof and poloxamer. In some embodiments, the long-acting formulation of rilpivirine or a pharmaceutically acceptable salt thereof comprises rilpivirine or a pharmaceutically acceptable salt thereof, citric acid, and poloxamer. In some embodiments, the long-acting formulation of rilpivirine or a pharmaceutically acceptable salt thereof comprises 300 mg/mL rilpivirine, 1 mg/mL citric acid monohydrate, 50 mg/mL poloxamer 338, and water for injection. In some embodiments, the long-acting formulation of rilpivirine or a pharmaceutically acceptable salt thereof comprises 300 mg/mL rilpivirine, 1 mg/mL citric acid monohydrate, 50 mg/mL poloxamer 338, water for injection, glucose monohydrate, sodium dihydrogen phosphate monohydrate, and sodium hydroxide to adjust pH. In some embodiments, the rilpivirine or a pharmaceutically acceptable salt thereof in the long-acting formulation is in the form of the rilpivirine free base.

In some embodiments, the injections of cabotegravir or a pharmaceutically acceptable salt thereof are formulated as suspensions and the cabotegravir or a pharmaceutically acceptable salt thereof is in the free form (i.e., the non-salt form), and the injections of rilpivirine or a pharmaceutically acceptable salt thereof are formulated as suspensions and the rilpivirine or a pharmaceutically acceptable salt thereof is in the free form (i.e., the non-salt form). In some embodiments, the injections of cabotegravir or a pharmaceutically acceptable salt thereof are formulated as suspensions and the cabotegravir or a pharmaceutically acceptable salt thereof is in free acid/base form, and the injections of rilpivirine or a pharmaceutically acceptable salt thereof are formulated as suspensions and the rilpivirine or a pharmaceutically acceptable salt thereof is in free base form.

### Exemplary Dosing Regimens.

In some embodiments, a human is in a weight band of 35 kg to less than 40 kg, and the cabotegravir or a pharmaceutically acceptable salt thereof is administered as a 400 mg to 600 mg intramuscular injection, and the rilpivirine or a pharmaceutically acceptable salt thereof is administered as a 600 mg to 900 mg intramuscular injection. In some embodiments, the human weighs 35 kg to less than 40 kg, the loading dose of cabotegravir or a pharmaceutically acceptable salt thereof is administered as a 600 mg intramuscular injection, and the loading dose of rilpivirine or a pharmaceutically acceptable salt thereof is administered as a 900 mg intramuscular injection. In some embodiments, the human weighs 35 kg to less than 40 kg, the one or more maintenance doses of cabotegravir or a pharmaceutically acceptable salt thereof are administered as 400 mg intramuscular injections and the one or more maintenance doses of rilpivirine or a pharmaceutically acceptable salt thereof are administered as 600 mg intramuscular injections. In some embodiments, the one or more maintenance doses are administered once every 4 weeks ±7 days or less frequently. In some embodiments, the one or more maintenance doses are administered once every month ±7 days or once every 4 weeks ±7 days. In some embodiments, the one or more maintenance doses are administered once every month ±7 days. In some embodiments, the one or more maintenance doses are administered once every 4 weeks ±7 days. In some embodiments, the one or more maintenance doses are administered once every two months ±7 days or once every 8 weeks ±7 days. In some embodiments, the one or more maintenance doses are administered once every two months ±7 days. In some embodiments, the one or more maintenance doses are administered every 8 weeks ±7 days. In some embodiments, the one or more maintenance doses are administered once every month ±7 days or once every 4 weeks ±7 days, followed by once every two months ±7 days or once every 8 weeks ±7 days. In some embodiments, the one or more maintenance doses are administered once every month ±7 days or once every 4 weeks ±7 days for 1, 2, 3, 4, 5, 6, 7, 8, 9, 10, 11, 12, 13, 14, 15, 16, 17, 18, 19, 20, 21, 22, 23, or 24 times, followed by once every two months ±7 days or once every 8 weeks ±7 days. In some embodiments, the one or more maintenance doses are administered once every month ±7 days or once every 4 weeks ±7 days for 1, 2, 3, 4, 5, 6, 7, 8, 9, 10, 11, 12, 13, 14, 15, 16, 17, 18, 19, 20, 21, 22, 23, or 24 times, followed by once every two months ±7 days or once every 8 weeks ±7 days for at least for 1, 2, 3, 4, 5, 6, 7, 8, 9, 10, 11, 12, 13, 14, 15, 16, 17, 18, 19, 20, 21, 22, 23, or 24 times.

In some embodiments, the human in a weight band of 35 kg to less than 40 kg is further administered an oral lead-in dose. Thus, in some embodiments, the method or use further comprises orally administering lead-in doses of cabotegravir or a pharmaceutically acceptable salt thereof and rilpivirine or a pharmaceutically acceptable salt thereof prior to the loading dose, wherein the lead-in dose of cabotegravir or a pharmaceutically acceptable salt thereof is a once-daily dose of 30 mg acid equivalent and the lead-in dose of rilpivirine is a once-daily dose of 25 mg base equivalent. In some embodiments, the lead-in doses of cabotegravir or a pharmaceutically acceptable salt thereof and rilpivirine or a pharmaceutically acceptable salt thereof are administered once daily for at least 4 weeks, in particular once daily for 4 weeks. In some embodiments, the lead-in doses of cabotegravir or a pharmaceutically acceptable salt thereof are in the form of cabotegravir sodium. In some embodiments, the lead-in doses of rilpivirine or a pharmaceutically acceptable salt thereof are provided by a pharmaceutically acceptable salt of rilpivirine. In some embodiments, the pharmaceutically acceptable salt of rilpivirine is rilpivirine hydrochloride.

In some embodiments, the human is in a weight band of 25 kg to 34.9 kg, and the cabotegravir or a pharmaceutically acceptable salt thereof is administered as a 200 mg to 300 mg intramuscular injection, and the rilpivirine or a pharmaceutically acceptable salt thereof is administered as a 450 to 600 mg intramuscular injection. In some embodiments, the human weighs 25 kg to 34.9 kg, the loading dose of cabotegravir or a pharmaceutically acceptable salt thereof is administered as a 300 mg intramuscular injection, and the loading dose of rilpivirine or a pharmaceutically acceptable salt thereof is administered as a 600 mg intramuscular injection. In some embodiments, the human weighs 25 kg to 34.9 kg, the one or more maintenance doses of cabotegravir or a pharmaceutically acceptable salt thereof are administered as 200 mg intramuscular injections and the one or more maintenance doses of rilpivirine or a pharmaceutically acceptable salt thereof are administered as 450 mg intramuscular injections. In some embodiments, the one or more maintenance doses are administered once every 4 weeks ±7 days or less frequently. In some embodiments, the one or more maintenance doses are administered once every month ±7 days or once every 4 weeks ±7 days. In some embodiments, the one or more maintenance doses are administered once every month ±7 days. In some embodiments, the one or more maintenance doses are administered once every 4 weeks ±7 days. In some embodiments, the one or more maintenance doses are administered once every two months ±7 days or once every 8 weeks ±7 days. In some embodiments, the one or more maintenance doses are administered once every two months ±7 days. In some embodiments, the one or more maintenance doses are administered every 8 weeks ±7 days. In some embodiments, the one or more maintenance doses are administered once every month ±7 days or once every 4 weeks ±7 days, followed by once every two months ±7 days or once every 8 weeks ±7 days. In some embodiments, the one or more maintenance doses are administered once every month ±7 days or once every 4 weeks ±7 days for 1, 2, 3, 4, 5, 6, 7, 8, 9, 10, 11, 12, 13, 14, 15, 16, 17, 18, 19, 20, 21, 22, 23, or 24 times, followed by once every two months ±7 days or once every 8 weeks ±7 days. In some embodiments, the one or more maintenance doses are administered once every month ±7 days or once every 4 weeks ±7 days for 1, 2, 3, 4, 5, 6, 7, 8, 9, 10, 11, 12, 13, 14, 15, 16, 17, 18, 19, 20, 21, 22, 23, or 24 times, followed by once every two months ±7 days or once every 8 weeks ±7 days for at least for 1, 2, 3, 4, 5, 6, 7, 8, 9, 10, 11, 12, 13, 14, 15, 16, 17, 18, 19, 20, 21, 22, 23, or 24 times.

In some embodiments, the human in a weight band of 25 kg to 34.9 kg is further administered an oral lead-in dose. Thus, in some embodiments, the method or use further comprises orally administering lead-in doses of cabotegravir or a pharmaceutically acceptable salt thereof and rilpivirine or a pharmaceutically acceptable salt thereof prior to the loading dose, wherein the lead-in dose of cabotegravir or a pharmaceutically acceptable salt thereof is a once-daily dose of 10 mg acid equivalent and the lead-in dose of rilpivirine is a once-daily dose of 25 mg base equivalent. In some embodiments, the lead-in doses of cabotegravir or a pharmaceutically acceptable salt thereof and rilpivirine or a pharmaceutically acceptable salt thereof are administered once daily for at least 4 weeks, in particular once daily for 4 weeks. In some embodiments, the lead-in doses of cabotegravir or a pharmaceutically acceptable salt thereof are administered as two 5 mg acid equivalent dispersible tablets. In some embodiments, the lead-in doses of rilpivirine or a pharmaceutically acceptable salt thereof are administered as one 25 mg base equivalent tablet. In some embodiments, the lead-in doses of cabotegravir or a pharmaceutically acceptable salt thereof are in the form of cabotegravir sodium. In some embodiments, the lead-in doses of rilpivirine or a pharmaceutically acceptable salt thereof are provided by a pharmaceutically acceptable salt of rilpivirine. In some embodiments, the pharmaceutically acceptable salt of rilpivirine is rilpivirine hydrochloride.

In some embodiments, the human is in a weight band of 20 kg to 24.9 kg, and the cabotegravir or a pharmaceutically acceptable salt thereof is administered as a 200 mg to 300 mg intramuscular injection, and the rilpivirine or a pharmaceutically acceptable salt thereof is administered as a 450 mg to 600 mg intramuscular injection. In some embodiments, the human weighs 20 kg to 24.9 kg, the loading dose of cabotegravir or a pharmaceutically acceptable salt thereof is administered as a 300 mg intramuscular injection, and the loading dose of rilpivirine or a pharmaceutically acceptable salt thereof is administered as a 600 mg intramuscular injection. In some embodiments, the human weighs 20 kg to 24.9 kg, the one or more maintenance doses of cabotegravir or a pharmaceutically acceptable salt thereof are administered as 200 mg intramuscular injections and the one or more maintenance doses of rilpivirine or a pharmaceutically acceptable salt thereof are administered as 450 mg intramuscular injections. In some embodiments, the one or more maintenance doses are administered once every 4 weeks ±7 days or less frequently. In some embodiments, the one or more maintenance doses are administered once every month ±7 days or once every 4 weeks ±7 days. In some embodiments, the one or more maintenance doses are administered once every month ±7 days. In some embodiments, the one or more maintenance doses are administered once every 4 weeks ±7 days. In some embodiments, the one or more maintenance doses are administered once every two months ±7 days or once every 8 weeks ±7 days. In some embodiments, the one or more maintenance doses are administered once every two months ±7 days. In some embodiments, the one or more maintenance doses are administered every 8 weeks ±7 days. In some embodiments, the one or more maintenance doses are administered once every month ±7 days or once every 4 weeks ±7 days, followed by once every two months ±7 days or once every 8 weeks ±7 days. In some embodiments, the one or more maintenance doses are administered once every month ±7 days or once every 4 weeks #7 days for 1, 2, 3, 4, 5, 6, 7, 8, 9, 10, 11, 12, 13, 14, 15, 16, 17, 18, 19, 20, 21, 22, 23, or 24 times, followed by once every two months ±7 days or once every 8 weeks ±7 days. In some embodiments, the one or more maintenance doses are administered once every month ±7 days or once every 4 weeks ±7 days for 1, 2, 3, 4, 5, 6, 7, 8, 9, 10, 11, 12, 13, 14, 15, 16, 17, 18, 19, 20, 21, 22, 23, or 24 times, followed by once every two months ±7 days or once every 8 weeks ±7 days for at least for 1, 2, 3, 4, 5, 6, 7, 8, 9, 10, 11, 12, 13, 14, 15, 16, 17, 18, 19, 20, 21, 22, 23, or 24 times.

In some embodiments, the human in a weight band of 20 kg to 24.9 kg is further administered an oral lead-in dose. Thus, in some embodiments, the method or use further comprises orally administering lead-in doses of cabotegravir or a pharmaceutically acceptable salt thereof and rilpivirine or a pharmaceutically acceptable salt thereof prior to the loading dose, wherein the lead-in dose of cabotegravir or a pharmaceutically acceptable salt thereof is a once-daily dose of 10 mg acid equivalent and the lead-in dose of rilpivirine or a pharmaceutically acceptable salt thereof is a once-daily dose of 15 mg base equivalent. In some embodiments, the lead-in doses of cabotegravir or a pharmaceutically acceptable salt thereof and rilpivirine or a pharmaceutically acceptable salt thereof are administered once daily for at least 4 weeks, in particular once daily for 4 weeks. In some embodiments, the lead-in doses of cabotegravir or a pharmaceutically acceptable salt thereof are administered as two 5 mg acid equivalent dispersible tablets. In some embodiments, the lead-in doses of rilpivirine or a pharmaceutically acceptable salt thereof are administered as six 2.5 mg base equivalent tablets, preferably as tablets dispersed in liquid. In some embodiments, the lead-in doses of cabotegravir or a pharmaceutically acceptable salt thereof are in the form of cabotegravir sodium. In some embodiments, the lead-in doses of rilpivirine or a pharmaceutically acceptable salt thereof are provided by a pharmaceutically acceptable salt of rilpivirine. In some embodiments, the pharmaceutically acceptable salt of rilpivirine is rilpivirine hydrochloride.

In some embodiments, the human is in a weight band of 14 kg to 19.9 kg, and the cabotegravir or a pharmaceutically acceptable salt thereof is administered as a 200 mg to 300 mg intramuscular injection, and the rilpivirine or a pharmaceutically acceptable salt thereof is administered as a 300 mg to 450 mg intramuscular injection. In some embodiments, the human weighs 14 kg to 19.9 kg, the loading dose of cabotegravir or a pharmaceutically acceptable salt thereof is administered as a 300 mg intramuscular injection, and the loading dose of rilpivirine or a pharmaceutically acceptable salt thereof is administered as a 450 mg intramuscular injection. In some embodiments, the human weighs 14 kg to 19.9 kg, the one or more maintenance doses of cabotegravir or a pharmaceutically acceptable salt thereof are administered as 200 mg intramuscular injections and the one or more maintenance doses of rilpivirine or a pharmaceutically acceptable salt thereof are administered as 300 mg intramuscular injections. In some embodiments, the one or more maintenance doses are administered once every 4 weeks ±7 days or less frequently. In some embodiments, the one or more maintenance doses are administered once every month ±7 days or once every 4 weeks ±7 days. In some embodiments, the one or more maintenance doses are administered once every month ±7 days. In some embodiments, the one or more maintenance doses are administered once every 4 weeks ±7 days. In some embodiments, the one or more maintenance doses are administered once every two months ±7 days or once every 8 weeks ±7 days. In some embodiments, the one or more maintenance doses are administered once every two months ±7 days. In some embodiments, the one or more maintenance doses are administered every 8 weeks ±7 days. In some embodiments, the one or more maintenance doses are administered once every month ±7 days or once every 4 weeks ±7 days, followed by once every two months ±7 days or once every 8 weeks ±7 days. In some embodiments, the one or more maintenance doses are administered once every month ±7 days or once every 4 weeks ±7 days for 1, 2, 3, 4, 5, 6, 7, 8, 9, 10, 11, 12, 13, 14, 15, 16, 17, 18, 19, 20, 21, 22, 23, or 24 times, followed by once every two months ±7 days or once every 8 weeks ±7 days. In some embodiments, the one or more maintenance doses are administered once every month ±7 days or once every 4 weeks ±7 days for 1, 2, 3, 4, 5, 6, 7, 8, 9, 10, 11, 12, 13, 14, 15, 16, 17, 18, 19, 20, 21, 22, 23, or 24 times, followed by once every two months ±7 days or once every 8 weeks ±7 days for at least for 1, 2, 3, 4, 5, 6, 7, 8, 9, 10, 11, 12, 13, 14, 15, 16, 17, 18, 19, 20, 21, 22, 23, or 24 times.

In some embodiments, the human in a weight band of 14 kg to 19.9 kg is further administered an oral lead-in dose. Thus, in some embodiments, the method or use further comprises orally administering lead-in doses of cabotegravir or a pharmaceutically acceptable salt thereof and rilpivirine or a pharmaceutically acceptable salt thereof prior to the loading dose, wherein the lead-in dose of cabotegravir or a pharmaceutically acceptable salt thereof is a once-daily dose of 10 mg acid equivalent and the lead-in dose of rilpivirine or a pharmaceutically acceptable salt thereof is a once-daily dose of 12.5 mg base equivalent. In some embodiments, the lead-in doses of cabotegravir or a pharmaceutically acceptable salt thereof and rilpivirine or a pharmaceutically acceptable salt thereof are administered once daily for at least 4 weeks, in particular once daily for 4 weeks. In some embodiments, the lead-in doses of cabotegravir or a pharmaceutically acceptable salt thereof are administered as two 5 mg acid equivalent dispersible tablets. In some embodiments, the lead-in doses of rilpivirine or a pharmaceutically acceptable salt thereof are administered as five 2.5 mg base equivalent tablets, preferably as tablets dispersed in liquid. In some embodiments, the lead-in doses of cabotegravir or a pharmaceutically acceptable salt thereof are in the form of cabotegravir sodium. In some embodiments, the lead-in doses of rilpivirine or a pharmaceutically acceptable salt thereof are provided by a pharmaceutically acceptable salt of rilpivirine. In some embodiments, the pharmaceutically acceptable salt of rilpivirine is rilpivirine hydrochloride.

In some embodiments, the human is in a weight band of 10 kg to 13.9 kg, and the cabotegravir or a pharmaceutically acceptable salt thereof is administered as a 200 mg to 300 mg intramuscular injection, and the rilpivirine or a pharmaceutically acceptable salt thereof is administered as a 300 mg to 450 mg intramuscular injection. In some embodiments, the human weighs 10 kg to 13.9 kg, the loading dose of cabotegravir or a pharmaceutically acceptable salt thereof is administered as a 300 mg intramuscular injection, and the loading dose of rilpivirine or a pharmaceutically acceptable salt thereof is administered as a 450 mg intramuscular injection. In some embodiments, the human weighs 10 kg to 13.9 kg, the one or more maintenance doses of cabotegravir or a pharmaceutically acceptable salt thereof are administered as 200 mg intramuscular injections and the one or more maintenance doses of rilpivirine or a pharmaceutically acceptable salt thereof are administered as 300 mg intramuscular injections. In some embodiments, the one or more maintenance doses are administered once every 4 weeks ±7 days or less frequently. In some embodiments, the one or more maintenance doses are administered once every month ±7 days or once every 4 weeks ±7 days. In some embodiments, the one or more maintenance doses are administered once every month ±7 days. In some embodiments, the one or more maintenance doses are administered once every 4 weeks ±7 days. In some embodiments, the one or more maintenance doses are administered once every two months ±7 days or once every 8 weeks ±7 days. In some embodiments, the one or more maintenance doses are administered once every two months ±7 days. In some embodiments, the one or more maintenance doses are administered every 8 weeks ±7 days. In some embodiments, the one or more maintenance doses are administered once every month ±7 days or once every 4 weeks ±7 days, followed by once every two months ±7 days or once every 8 weeks ±7 days. In some embodiments, the one or more maintenance doses are administered once every month ±7 days or once every 4 weeks ±7 days for 1, 2, 3, 4, 5, 6, 7, 8, 9, 10, 11, 12, 13, 14, 15, 16, 17, 18, 19, 20, 21, 22, 23, or 24 times, followed by once every two months ±7 days or once every 8 weeks ±7 days. In some embodiments, the one or more maintenance doses are administered once every month ±7 days or once every 4 weeks ±7 days for 1, 2, 3, 4, 5, 6, 7, 8, 9, 10, 11, 12, 13, 14, 15, 16, 17, 18, 19, 20, 21, 22, 23, or 24 times, followed by once every two months ±7 days or once every 8 weeks ±7 days for at least for 1, 2, 3, 4, 5, 6, 7, 8, 9, 10, 11, 12, 13, 14, 15, 16, 17, 18, 19, 20, 21, 22, 23, or 24 times.

In some embodiments, the human in a weight band of 10 kg to 13.9 kg is further administered an oral lead-in dose. Thus, in some embodiments, the method or use further comprises orally administering lead-in doses of cabotegravir or a pharmaceutically acceptable salt thereof and rilpivirine or a pharmaceutically acceptable salt thereof prior to the loading dose, wherein the lead-in dose of cabotegravir or a pharmaceutically acceptable salt thereof is a once-daily dose of 10 mg acid equivalent and the lead-in dose of rilpivirine or a pharmaceutically acceptable salt thereof is a once-daily dose of 12.5 mg base equivalent. In some embodiments, the lead-in doses of cabotegravir or a pharmaceutically acceptable salt thereof and rilpivirine or a pharmaceutically acceptable salt thereof are administered once daily for at least 4 weeks, in particular once daily for 4 weeks. In some embodiments, the lead-in doses of cabotegravir or a pharmaceutically acceptable salt thereof are administered as two 5 mg acid equivalent dispersible tablets. In some embodiments, the lead-in doses of rilpivirine or a pharmaceutically acceptable salt thereof are administered as five 2.5 mg base equivalent tablets, preferably as tablets dispersed in liquid. In some embodiments, the lead-in doses of cabotegravir or a pharmaceutically acceptable salt thereof are in the form of cabotegravir sodium. In some embodiments, the lead-in doses of rilpivirine or a pharmaceutically acceptable salt thereof are provided by a pharmaceutically acceptable salt of rilpivirine. In some embodiments, the pharmaceutically acceptable salt of rilpivirine is rilpivirine hydrochloride.

In some embodiments, the disclosure provides a method of treating HIV infection, the method comprising: administering to a human in need thereof a loading dose comprising (i) an intramuscular injection of cabotegravir or a pharmaceutically acceptable salt thereof and (ii) an intramuscular injection of rilpivirine or a pharmaceutically acceptable salt thereof; and thereafter administering one or more maintenance doses comprising (i) an intramuscular injection of cabotegravir or a pharmaceutically acceptable salt thereof and (ii) an intramuscular injection of rilpivirine or a pharmaceutically acceptable salt thereof; wherein the human is 2 years of age to less than 12 years of age; and wherein: (a) the human weighs 35 kg to less than 40 kg; the loading dose of cabotegravir or a pharmaceutically acceptable salt thereof is administered as a 600 mg intramuscular injection, and the loading dose of rilpivirine or a pharmaceutically acceptable salt thereof is administered as a 900 mg intramuscular injection; and the one or more maintenance doses of cabotegravir or a pharmaceutically acceptable salt thereof are administered as 400 mg intramuscular injections and the one or more maintenance doses of rilpivirine or a pharmaceutically acceptable salt thereof are administered as 600 mg intramuscular injections; (b) the human weighs 25 kg to 34.9 kg; the loading dose of cabotegravir or a pharmaceutically acceptable salt thereof is administered as a 300 mg intramuscular injection, and the loading dose of rilpivirine or a pharmaceutically acceptable salt thereof is administered as a 600 mg intramuscular injection; and the one or more maintenance doses of cabotegravir or a pharmaceutically acceptable salt thereof are administered as 200 mg intramuscular injections and the one or more maintenance doses of rilpivirine or a pharmaceutically acceptable salt thereof are administered as 450 mg intramuscular injections; (c) the human weighs 20 kg to 24.9 kg; the loading dose cabotegravir or a pharmaceutically acceptable salt thereof is administered as a 300 mg intramuscular injection and the loading dose of rilpivirine or a pharmaceutically acceptable salt thereof is administered as a 600 mg intramuscular injection; and the one or more maintenance doses of cabotegravir or a pharmaceutically acceptable salt thereof are administered as 200 mg intramuscular injections and the one or more maintenance doses of rilpivirine or a pharmaceutically acceptable salt thereof are administered as 450 mg intramuscular injections; (d) the human weighs 14 kg to 19.9 kg; the loading dose of cabotegravir or a pharmaceutically acceptable salt thereof is administered as a 300 mg intramuscular injection and the loading dose of rilpivirine or a pharmaceutically acceptable salt thereof is administered as a 450 mg intramuscular injection; and the one or more maintenance doses of cabotegravir or a pharmaceutically acceptable salt thereof are administered as 200 mg intramuscular injections and the one or more maintenance doses of rilpivirine or a pharmaceutically acceptable salt thereof are administered as 300 mg intramuscular injections; or (e) the human weighs 10 kg to 13.9 kg; the loading dose of cabotegravir or a pharmaceutically acceptable salt thereof is administered as a 300 mg intramuscular injection and the loading dose of rilpivirine or a pharmaceutically acceptable salt thereof is administered as a 450 mg intramuscular injection; and the one or more maintenance doses of cabotegravir or a pharmaceutically acceptable salt thereof are administered as 200 mg intramuscular injections and the one or more maintenance doses of rilpivirine or a pharmaceutically acceptable salt thereof are administered as 300 mg intramuscular injections.

In some embodiments, the human is further administered an oral lead-in dose. Thus, in some embodiments, the method further comprises orally administering lead-in doses of cabotegravir or a pharmaceutically acceptable salt thereof and rilpivirine or a pharmaceutically acceptable salt thereof prior to the loading dose, wherein (a) if the human weighs 35 kg to less than 40 kg, the lead-in dose of cabotegravir or a pharmaceutically acceptable salt thereof is a once-daily dose of 30 mg acid equivalent and the lead-in dose of rilpivirine or a pharmaceutically acceptable salt thereof is a once-daily dose of 25 mg base equivalent; (b) if the human weighs 25 kg to 34.9 kg, the lead-in dose of cabotegravir or a pharmaceutically acceptable salt thereof is a once-daily dose of 10 mg acid equivalent and the lead-in dose of rilpivirine or a pharmaceutically acceptable salt thereof is a once-daily dose of 25 mg base equivalent; (c) if the human weighs 20 kg to 24.9 kg, the lead-in dose of cabotegravir or a pharmaceutically acceptable salt thereof is a once-daily dose of 10 mg acid equivalent and the lead-in dose of rilpivirine or a pharmaceutically acceptable salt thereof is a once-daily dose of 15 mg base equivalent; (d) if the human weighs 14 kg to 19.9 kg, the lead-in dose of cabotegravir or a pharmaceutically acceptable salt thereof is a once-daily dose of 10 mg acid equivalent and the lead-in dose of rilpivirine or a pharmaceutically acceptable salt thereof is a once-daily dose of 12.5 mg base equivalent; or (e) if the human weighs 10 kg to 13.9 kg, the lead-in dose of cabotegravir or a pharmaceutically acceptable salt thereof is a once-daily dose of 10 mg acid equivalent and the lead-in dose of rilpivirine or a pharmaceutically acceptable salt thereof is a once-daily dose of 12.5 mg base equivalent.

In some embodiments, the disclosure provides the use of a loading dose comprising (i) cabotegravir or a pharmaceutically acceptable salt thereof and (ii) rilpivirine or a pharmaceutically acceptable salt thereof; and one or more maintenance doses comprising (i) cabotegravir or a pharmaceutically acceptable salt thereof and (ii) rilpivirine or a pharmaceutically acceptable salt thereof in the manufacture of a medicament for use in treatment of HIV infection in a human; wherein the human is 2 years of age to less than 12 years of age; wherein the medicament is formulated for intramuscular injections; and wherein: (a) the human weighs 35 kg to less than 40 kg; the loading dose of cabotegravir or a pharmaceutically acceptable salt thereof is a 600 mg intramuscular injection, and the loading dose of rilpivirine or a pharmaceutically acceptable salt thereof is a 900 mg intramuscular injection; and the one or more maintenance doses of cabotegravir or a pharmaceutically acceptable salt thereof are 400 mg intramuscular injections and the one or more maintenance doses of rilpivirine or a pharmaceutically acceptable salt thereof are 600 mg intramuscular injections; (b) the human weighs 25 kg to 34.9 kg; the loading dose of cabotegravir or a pharmaceutically acceptable salt thereof is a 300 mg intramuscular injection, and the loading dose of rilpivirine or a pharmaceutically acceptable salt thereof is a 600 mg intramuscular injection; and the one or more maintenance doses of cabotegravir or a pharmaceutically acceptable salt thereof are 200 mg intramuscular injections and the one or more maintenance doses of rilpivirine or a pharmaceutically acceptable salt thereof are 450 mg intramuscular injections; (c) the human weighs 20 kg to 24.9 kg; the loading dose cabotegravir or a pharmaceutically acceptable salt thereof is a 300 mg intramuscular injection and the loading dose of rilpivirine or a pharmaceutically acceptable salt thereof is a 600 mg intramuscular injection; and the one or more maintenance doses of cabotegravir or a pharmaceutically acceptable salt thereof are 200 mg intramuscular injections and the one or more maintenance doses of rilpivirine or a pharmaceutically acceptable salt thereof are 450 mg intramuscular injections; (d) the human weighs 14 kg to 19.9 kg; the loading dose of cabotegravir or a pharmaceutically acceptable salt thereof is a 300 mg intramuscular injection and the loading dose of rilpivirine or a pharmaceutically acceptable salt thereof is a 450 mg intramuscular injection; and the one or more maintenance doses of cabotegravir or a pharmaceutically acceptable salt thereof are 200 mg intramuscular injections and the one or more maintenance doses of rilpivirine or a pharmaceutically acceptable salt thereof are 300 mg intramuscular injections; or (e) the human weighs 10 kg to 13.9 kg; the loading dose of cabotegravir or a pharmaceutically acceptable salt thereof is a 300 mg intramuscular injection and the loading dose of rilpivirine or a pharmaceutically acceptable salt thereof is a 450 mg intramuscular injection; and the one or more maintenance doses of cabotegravir or a pharmaceutically acceptable salt thereof are 200 mg intramuscular injections and the one or more maintenance doses of rilpivirine or a pharmaceutically acceptable salt thereof are 300 mg intramuscular injections.

In some embodiments, the disclosure provides a loading dose comprising (i) cabotegravir or a pharmaceutically acceptable salt thereof and (ii) rilpivirine or a pharmaceutically acceptable salt thereof; and one or more maintenance doses comprising (i) cabotegravir or a pharmaceutically acceptable salt thereof and (ii) rilpivirine or a pharmaceutically acceptable salt thereof for use in treatment of HIV infection in a human; wherein the human is 2 years of age to less than 12 years of age; wherein the cabotegravir or a pharmaceutically acceptable salt thereof and rilpivirine or a pharmaceutically acceptable salt are formulated for intramuscular injection; and wherein: (a) the human weighs 35 kg to less than 40 kg; the loading dose of cabotegravir or a pharmaceutically acceptable salt thereof is a 600 mg intramuscular injection, and the loading dose of rilpivirine or a pharmaceutically acceptable salt thereof is a 900 mg intramuscular injection; and the one or more maintenance doses of cabotegravir or a pharmaceutically acceptable salt thereof are 400 mg intramuscular injections and the one or more maintenance doses of rilpivirine or a pharmaceutically acceptable salt thereof are 600 mg intramuscular injections; (b) the human weighs 25 kg to 34.9 kg; the loading dose of cabotegravir or a pharmaceutically acceptable salt thereof is a 300 mg intramuscular injection, and the loading dose of rilpivirine or a pharmaceutically acceptable salt thereof is a 600 mg intramuscular injection; and the one or more maintenance doses of cabotegravir or a pharmaceutically acceptable salt thereof are 200 mg intramuscular injections and the one or more maintenance doses of rilpivirine or a pharmaceutically acceptable salt thereof are 450 mg intramuscular injections; (c) the human weighs 20 kg to 24.9 kg; the loading dose cabotegravir or a pharmaceutically acceptable salt thereof is a 300 mg intramuscular injection and the loading dose of rilpivirine or a pharmaceutically acceptable salt thereof is a 600 mg intramuscular injection; and the one or more maintenance doses of cabotegravir or a pharmaceutically acceptable salt thereof are 200 mg intramuscular injections and the one or more maintenance doses of rilpivirine or a pharmaceutically acceptable salt thereof are 450 mg intramuscular injections; (d) the human weighs 14 kg to 19.9 kg; the loading dose of cabotegravir or a pharmaceutically acceptable salt thereof is a 300 mg intramuscular injection and the loading dose of rilpivirine or a pharmaceutically acceptable salt thereof is a 450 mg intramuscular injection; and the one or more maintenance doses of cabotegravir or a pharmaceutically acceptable salt thereof are 200 mg intramuscular injections and the one or more maintenance doses of rilpivirine or a pharmaceutically acceptable salt thereof are 300 mg intramuscular injections; or (e) the human weighs 10 kg to 13.9 kg; the loading dose of cabotegravir or a pharmaceutically acceptable salt thereof is a 300 mg intramuscular injection and the loading dose of rilpivirine or a pharmaceutically acceptable salt thereof is a 450 mg intramuscular injection; and the one or more maintenance doses of cabotegravir or a pharmaceutically acceptable salt thereof are 200 mg intramuscular injections and the one or more maintenance doses of rilpivirine or a pharmaceutically acceptable salt thereof are 300 mg intramuscular injections.

In some embodiments, the disclosure provides cabotegravir or a pharmaceutically acceptable salt thereof indicated in combination with rilpivirine or a pharmaceutically acceptable salt thereof for use in treatment of HIV infection in a human; wherein the human is 2 years of age to less than 12 years of age; and wherein: (a) the human weighs 35 kg to less than 40 kg; and the cabotegravir or a pharmaceutically acceptable salt thereof is formulated as a 600 mg intramuscular injection; (b) the human weighs 25 kg to 34.9 kg; and the cabotegravir or a pharmaceutically acceptable salt thereof is formulated as a 300 mg intramuscular injection; (c) the human weighs 20 kg to 24.9 kg; and the cabotegravir or a pharmaceutically acceptable salt thereof is formulated as a 300 mg intramuscular injection; (d) the human weighs 14 kg to 19.9 kg; and the cabotegravir or a pharmaceutically acceptable salt thereof is formulated as a 300 mg intramuscular injection; or (e) the human weighs 10 kg to 13.9 kg; and the cabotegravir or a pharmaceutically acceptable salt thereof is formulated as a 300 mg intramuscular injection.

In some embodiments, the disclosure provides cabotegravir or a pharmaceutically acceptable salt thereof indicated in combination with rilpivirine or a pharmaceutically acceptable salt thereof for use in treatment of HIV infection in a human; wherein the human is 2 years of age to less than 12 years of age; and wherein: (a) the human weighs 35 kg to less than 40 kg; and the cabotegravir or a pharmaceutically acceptable salt thereof is formulated as a 400 mg intramuscular injection; (b) the human weighs 25 kg to 34.9 kg; and the cabotegravir or a pharmaceutically acceptable salt thereof is formulated as a 200 mg intramuscular injection; (c) the human weighs 20 kg to 24.9 kg; and the cabotegravir or a pharmaceutically acceptable salt thereof is formulated as a 200 mg intramuscular injection; (d) the human weighs 14 kg to 19.9 kg; and the cabotegravir or a pharmaceutically acceptable salt thereof is formulated as a 200 mg intramuscular injection; or (e) the human weighs 10 kg to 13.9 kg; and the cabotegravir or a pharmaceutically acceptable salt thereof is formulated as a 200 mg intramuscular injection.

In some embodiments, the disclosure provides rilpivirine or a pharmaceutically acceptable salt thereof indicated in combination with cabotegravir or a pharmaceutically acceptable salt thereof for use in treatment of HIV infection in a human; wherein the human is 2 years of age to less than 12 years of age; and wherein: (a) the human weighs 35 kg to less than 40 kg; and the rilpivirine or a pharmaceutically acceptable salt thereof is formulated as a 900 mg intramuscular injection; (b) the human weighs 25 kg to 34.9 kg; and the rilpivirine or a pharmaceutically acceptable salt thereof is formulated as a 600 mg intramuscular injection; (c) the human weighs 20 kg to 24.9 kg; and the rilpivirine or a pharmaceutically acceptable salt thereof is formulated as a 600 mg intramuscular injection; (d) the human weighs 14 kg to 19.9 kg; and the rilpivirine or a pharmaceutically acceptable salt thereof is formulated as a 450 mg intramuscular injection; or (e) the human weighs 10 kg to 13.9 kg; and the rilpivirine or a pharmaceutically acceptable salt thereof is formulated as a 450 mg intramuscular injection.

In some embodiments, the disclosure provides rilpivirine or a pharmaceutically acceptable salt thereof indicated in combination with cabotegravir or a pharmaceutically acceptable salt thereof for use in treatment of HIV infection in a human; wherein the human is 2 years of age to less than 12 years of age; and wherein: (a) the human weighs 35 kg to less than 40 kg; and the rilpivirine or a pharmaceutically acceptable salt thereof is formulated as a 600 mg intramuscular injection; (b) the human weighs 25 kg to 34.9 kg; and the rilpivirine or a pharmaceutically acceptable salt thereof is formulated as a 450 mg intramuscular injection; (c) the human weighs 20 kg to 24.9 kg; and the rilpivirine or a pharmaceutically acceptable salt thereof is formulated as a 450 mg intramuscular injection; (d) the human weighs 14 kg to 19.9 kg; and the rilpivirine or a pharmaceutically acceptable salt thereof is formulated as a 300 mg intramuscular injection; or (e) the human weighs 10 kg to 13.9 kg; and the rilpivirine or a pharmaceutically acceptable salt thereof is formulated as a 300 mg intramuscular injection.

In some embodiments, the use further comprises an oral lead-in dose. Thus, in some embodiments, the use further comprises oral lead-in doses of cabotegravir or a pharmaceutically acceptable salt thereof and rilpivirine or a pharmaceutically acceptable salt thereof prior to the loading dose, wherein (a) if the human weighs 35 kg to less than 40 kg, the lead-in dose of cabotegravir or a pharmaceutically acceptable salt thereof is a once-daily dose of 30 mg acid equivalent and the lead-in dose of rilpivirine or a pharmaceutically acceptable salt thereof is a once-daily dose of 25 mg base equivalent; (b) if the human weighs 25 kg to 34.9 kg, the lead-in dose of cabotegravir or a pharmaceutically acceptable salt thereof is a once-daily dose of 10 mg acid equivalent and the lead-in dose of rilpivirine or a pharmaceutically acceptable salt thereof is a once-daily dose of 25 mg base equivalent; (c) if the human weighs 20 kg to 24.9 kg, the lead-in dose of cabotegravir or a pharmaceutically acceptable salt thereof is a once-daily dose of 10 mg acid equivalent and the lead-in dose of rilpivirine or a pharmaceutically acceptable salt thereof is a once-daily dose of 15 mg base equivalent; (d) if the human weighs 14 kg to 19.9 kg, the lead-in dose of cabotegravir or a pharmaceutically acceptable salt thereof is a once-daily dose of 10 mg acid equivalent and the lead-in dose of rilpivirine or a pharmaceutically acceptable salt thereof is a once-daily dose of 12.5 mg base equivalent; or (e) if the human weighs 10 kg to 13.9 kg, the lead-in dose of cabotegravir or a pharmaceutically acceptable salt thereof is a once-daily dose of 10 mg acid equivalent and the lead-in dose of rilpivirine or a pharmaceutically acceptable salt thereof is a once-daily dose of 12.5 mg base equivalent.

### Numbered Embodiments

Embodiment 1. A method of treating HIV infection, the method comprising: administering to a human in need thereof a loading dose comprising (i) an intramuscular injection of cabotegravir or a pharmaceutically acceptable salt thereof and (ii) an intramuscular injection of rilpivirine or a pharmaceutically acceptable salt thereof; and
thereafter administering one or more maintenance doses comprising (i) an intramuscular injection of cabotegravir or a pharmaceutically acceptable salt thereof and (ii) an intramuscular injection of rilpivirine or a pharmaceutically acceptable salt thereof;
wherein the human is 2 years of age to less than 12 years of age.

Embodiment 2. The method of embodiment 1, wherein the human weighs 10 kg to less than 40 kg, preferably 10 kg to 34.9 kg.

Embodiment 3. The method of embodiment 1 or 2, wherein the loading dose of cabotegravir or a pharmaceutically acceptable salt thereof is greater than the one or more maintenance doses of cabotegravir or a pharmaceutically acceptable salt thereof.

Embodiment 4. The method of any one of the preceding embodiments, wherein the loading dose of rilpivirine or a pharmaceutically acceptable salt thereof is greater than the one or more maintenance doses of rilpivirine or a pharmaceutically acceptable salt thereof.

Embodiment 5. The method of any one of the preceding embodiments, wherein the cabotegravir or a pharmaceutically acceptable salt thereof is administered as a 50 mg to 600 mg intramuscular injection, preferably a 100 mg to 600 mg intramuscular injection, a 150 mg to 600 mg intramuscular injection, a 200 mg to 600 mg intramuscular injection, a 300 mg to 600 mg intramuscular injection, or a 200 mg to 400 mg intramuscular injection.

Embodiment 6. The method of any one of the preceding embodiments, wherein the rilpivirine or a pharmaceutically acceptable salt thereof is administered as a 50 mg to 900 mg intramuscular injection, preferably a 100 mg to 900 mg intramuscular injection, a 150 mg to 900 mg intramuscular injection, a 200 mg to 900 mg intramuscular injection, a 250 mg to 900 mg intramuscular injection, a 300 mg to 900 mg intramuscular injection, a 450 mg to 900 mg intramuscular injection, or a 300 mg to 600 mg intramuscular injection.

Embodiment 7. The method of any one of the preceding embodiments, wherein the one or more maintenance doses are administered at a regular interval.

Embodiment 8. The method of any one of the preceding embodiments, wherein the one or more maintenance doses are administered once every 4 weeks ±7 days or less frequently.

Embodiment 9. The method of embodiment 8, wherein the one or more maintenance doses are administered once every month ±7 days or once every 4 weeks ±7 days.

Embodiment 10. The method of embodiment 9, wherein the one or more maintenance doses are administered once every month ±7 days.

Embodiment 11. The method of embodiment 9, wherein the one or more maintenance doses are administered once every 4 weeks ±7 days.

Embodiment 12. The method of embodiment 8, wherein the one or more maintenance doses are administered once every two months ±7 days or once every 8 weeks ±7 days.

Embodiment 13. The method of embodiment 12, wherein the one or more maintenance doses are administered once every two months ±7 days.

Embodiment 14. The method of embodiment 12, wherein the one or more maintenance doses are administered every 8 weeks ±7 days.

Embodiment 15. The method of embodiment 8, wherein the one or more maintenance doses are administered once every month ±7 days or once every 4 weeks ±7 days, followed by once every two months ±7 days or once every 8 weeks ±7 days.

Embodiment 16. The method of embodiment 15, wherein the one or more maintenance doses are administered once every month ±7 days or once every 4 weeks ±7 days for 1, 2, 3, 4, 5, 6, 7, 8, 9, 10, 11, 12, 13, 14, 15, 16, 17, 18, 19, 20, 21, 22, 23, or 24 times, followed by once every two months ±7 days or once every 8 weeks ±7 days.

Embodiment 17. The method of embodiment 1, wherein the human weighs 35 kg to less than 40 kg, the cabotegravir or a pharmaceutically acceptable salt thereof is administered as a 400 mg to 600 mg intramuscular injection, and the rilpivirine or a pharmaceutically acceptable salt thereof is administered as a 600 mg to 900 mg intramuscular injection.

Embodiment 18. The method of embodiment 17, wherein the loading dose of cabotegravir or a pharmaceutically acceptable salt thereof is administered as a 600 mg intramuscular injection, and the loading dose of rilpivirine or a pharmaceutically acceptable salt thereof is administered as a 900 mg intramuscular injection.

Embodiment 19. The method of embodiment 18, wherein the one or more maintenance doses of cabotegravir or a pharmaceutically acceptable salt thereof are administered as 400 mg intramuscular injections and the one or more maintenance doses of rilpivirine or a pharmaceutically acceptable salt thereof are administered as 600 mg intramuscular injections.

Embodiment 20. The method of embodiment 1, wherein the human weighs 25 kg to 34.9 kg, the cabotegravir or a pharmaceutically acceptable salt thereof is administered as a 200 mg to 300 mg intramuscular injection, and the rilpivirine or a pharmaceutically acceptable salt thereof is administered as a 450 to 600 mg intramuscular injection.

Embodiment 21. The method of embodiment 20, wherein the loading dose of cabotegravir or a pharmaceutically acceptable salt thereof is administered as a 300 mg intramuscular injection, and the loading dose of rilpivirine or a pharmaceutically acceptable salt thereof is administered as a 600 mg intramuscular injection.

Embodiment 22. The method of embodiment 21, wherein the one or more maintenance doses of cabotegravir or a pharmaceutically acceptable salt thereof are administered as 200 mg intramuscular injections and the one or more maintenance doses of rilpivirine or a pharmaceutically acceptable salt thereof are administered as 450 mg intramuscular injections.

Embodiment 23. The method of embodiment 1, wherein the human weighs 20 kg to 24.9 kg, the cabotegravir or a pharmaceutically acceptable salt thereof is administered as a 200 mg to 300 mg intramuscular injection, and the rilpivirine or a pharmaceutically acceptable salt thereof is administered as a 450 mg to 600 mg intramuscular injection.

Embodiment 24. The method of embodiment 23, wherein the loading dose of cabotegravir or a pharmaceutically acceptable salt thereof is administered as a 300 mg intramuscular injection, and the loading dose of rilpivirine or a pharmaceutically acceptable salt thereof is administered as a 600 mg intramuscular injection.

Embodiment 25. The method of embodiment 24, wherein the one or more maintenance doses of cabotegravir or a pharmaceutically acceptable salt thereof are administered as 200 mg intramuscular injections and the one or more maintenance doses of rilpivirine or a pharmaceutically acceptable salt thereof are administered as 450 mg intramuscular injections.

Embodiment 26. The method of embodiment 1, wherein the human weighs 14 kg to 19.9 kg, the cabotegravir or a pharmaceutically acceptable salt thereof is administered as a 200 mg to 300 mg intramuscular injection, and the rilpivirine or a pharmaceutically acceptable salt thereof is administered as a 300 mg to 450 mg intramuscular injection.

Embodiment 27. The method of embodiment 26, wherein the loading dose of cabotegravir or a pharmaceutically acceptable salt thereof is administered as a 300 mg intramuscular injection, and the loading dose of rilpivirine or a pharmaceutically acceptable salt thereof is administered as a 450 mg intramuscular injection.

Embodiment 28. The method of embodiment 27, wherein the one or more maintenance doses of cabotegravir or a pharmaceutically acceptable salt thereof are administered as 200 mg intramuscular injections and the one or more maintenance doses of rilpivirine or a pharmaceutically acceptable salt thereof are administered as 300 mg intramuscular injections.

Embodiment 29. The method of embodiment 1, wherein the human weighs 10 kg to 13.9 kg, the cabotegravir or a pharmaceutically acceptable salt thereof is administered as a 200 mg to 300 mg intramuscular injection, and the rilpivirine or a pharmaceutically acceptable salt thereof is administered as a 300 mg to 450 mg intramuscular injection.

Embodiment 30. The method of embodiment 29, wherein the loading dose of cabotegravir or a pharmaceutically acceptable salt thereof is administered as a 300 mg intramuscular injection, and the loading dose of rilpivirine or a pharmaceutically acceptable salt thereof is administered as a 450 mg intramuscular injection.

Embodiment 31. The method of embodiment 30, wherein the one or more maintenance doses of cabotegravir or a pharmaceutically acceptable salt thereof are administered as 200 mg intramuscular injections and the one or more maintenance doses of rilpivirine or a pharmaceutically acceptable salt thereof are administered as 300 mg intramuscular injections.

Embodiment 32. The method of any one of embodiments 17 to 31, wherein the one or more maintenance doses are administered once every 4 weeks ±7 days or less frequently.

Embodiment 33. The method of embodiment 32, wherein the one or more maintenance doses are administered once every month ±7 days or once every 4 weeks ±7 days.

Embodiment 34. The method of embodiment 33, wherein the one or more maintenance doses are administered once every month ±7 days.

Embodiment 35. The method of embodiment 33, wherein the one or more maintenance doses are administered once every 4 weeks ±7 days.

Embodiment 36. The method of embodiment 32, wherein the one or more maintenance doses are administered once every two months ±7 days or once every 8 weeks ±7 days.

Embodiment 37. The method of embodiment 36, wherein the one or more maintenance doses are administered once every two months ±7 days.

Embodiment 38. The method of embodiment 36, wherein the one or more maintenance doses are administered every 8 weeks ±7 days.

Embodiment 39. The method of any one of embodiments 19, 22, 25, 28, or 31, wherein the one or more maintenance doses are administered once every month ±7 days or once every 4 weeks ±7 days, followed by once every two months ±7 days or once every 8 weeks ±7 days.

Embodiment 40. The method of embodiment 39, wherein the one or more maintenance doses are administered once every month ±7 days or once every 4 weeks ±7 days for 1, 2, 3, 4, 5, 6, 7, 8, 9, 10, 11, 12, 13, 14, 15, 16, 17, 18, 19, 20, 21, 22, 23, or 24 times, followed by once every two months ±7 days or once every 8 weeks ±7 days.

Embodiment 41. The method of any one of the preceding embodiments, wherein the loading dose of cabotegravir or a pharmaceutically acceptable salt thereof and the loading dose of rilpivirine or a pharmaceutically acceptable salt thereof are administered less than 1 hour apart, preferably less than 30 minutes apart.

Embodiment 42. The method of any one of the preceding embodiments, wherein for each maintenance dose of cabotegravir or a pharmaceutically acceptable salt thereof and rilpivirine or a pharmaceutically acceptable salt thereof, the cabotegravir or a pharmaceutically acceptable salt thereof and the rilpivirine or a pharmaceutically acceptable salt thereof are administered less than 1 hour apart, preferably less than 30 minutes apart.

Embodiment 43. The method of any one of the preceding embodiments, wherein the intramuscular injections are administered at a site selected from a gluteus medius or a lateral aspect of a thigh.

Embodiment 44. The method of any one of the preceding embodiments, wherein the injections of cabotegravir or a pharmaceutically acceptable salt thereof and rilpivirine or a pharmaceutically acceptable salt thereof are in the form of long-acting formulations.

Embodiment 45. The method of embodiment 44, wherein the long-acting formulation of cabotegravir or a pharmaceutically acceptable salt thereof comprises cabotegravir or a pharmaceutically acceptable salt thereof, polysorbate, and polyethylene glycol.

Embodiment 46. The method of embodiment 44 or 45, wherein the long-acting formulation of rilpivirine or a pharmaceutically acceptable salt thereof comprises rilpivirine or a pharmaceutically acceptable salt thereof and poloxamer.

Embodiment 47. The method of any one of the preceding embodiments, further comprising orally administering lead-in doses of cabotegravir or a pharmaceutically acceptable salt thereof and rilpivirine or a pharmaceutically acceptable salt thereof prior to the loading dose.

Embodiment 48. The method of embodiment 47, wherein the lead-in doses of cabotegravir or a pharmaceutically acceptable salt thereof are daily doses of 5 mg to 30 mg acid equivalent, preferably 10 mg to 30 mg acid equivalent.

Embodiment 49. The method of embodiment 48, wherein the lead-in doses of rilpivirine or a pharmaceutically acceptable salt thereof are daily doses of 2.5 mg to 25 mg base equivalent, preferably 12.5 mg to 25 mg base equivalent.

Embodiment 50. The method of any one of embodiments 17 to 19, further comprising orally administering lead-in doses of cabotegravir or a pharmaceutically acceptable salt thereof and rilpivirine or a pharmaceutically acceptable salt thereof prior to the loading dose, wherein the lead-in dose of cabotegravir or a pharmaceutically acceptable salt thereof is a once-daily dose of 30 mg acid equivalent and the lead-in dose of rilpivirine is a once-daily dose of 25 mg base equivalent.

Embodiment 51. The method of embodiment 50, wherein the lead-in doses of cabotegravir or a pharmaceutically acceptable salt thereof and rilpivirine or a pharmaceutically acceptable salt thereof are administered once daily for at least 4 weeks, in particular once daily for 4 weeks.

Embodiment 52. The method of embodiment 50 or 51, wherein the lead-in doses of rilpivirine or a pharmaceutically acceptable salt thereof are provided by a pharmaceutically acceptable salt of rilpivirine.

Embodiment 53. The method of embodiment 52, wherein the pharmaceutically acceptable salt of rilpivirine is rilpivirine hydrochloride.

Embodiment 54. The method of any one of embodiments 20 to 22, further comprising orally administering lead-in doses of cabotegravir or a pharmaceutically acceptable salt thereof and rilpivirine or a pharmaceutically acceptable salt thereof prior to the loading dose, wherein the lead-in dose of cabotegravir or a pharmaceutically acceptable salt thereof is a once-daily dose of 10 mg acid equivalent and the lead-in dose of rilpivirine is a once-daily dose of 25 mg base equivalent.

Embodiment 55. The method of embodiment 54, wherein the lead-in doses of cabotegravir or a pharmaceutically acceptable salt thereof and rilpivirine or a pharmaceutically acceptable salt thereof are administered once daily for at least 4 weeks, in particular once daily for 4 weeks.

Embodiment 56. The method of embodiment 54 or 55, wherein the lead-in doses of cabotegravir or a pharmaceutically acceptable salt thereof are administered as two 5 mg acid equivalent dispersible tablets.

Embodiment 57. The method of any one of embodiments 54 to 56, wherein the lead-in doses of rilpivirine or a pharmaceutically acceptable salt thereof are provided by a pharmaceutically acceptable salt of rilpivirine.

Embodiment 58. The method of embodiment 57, wherein the pharmaceutically acceptable salt of rilpivirine is rilpivirine hydrochloride.

Embodiment 59. The method of any one of embodiments 23 to 25, further comprising orally administering lead-in doses of cabotegravir or a pharmaceutically acceptable salt thereof and rilpivirine or a pharmaceutically acceptable salt thereof prior to the loading dose, wherein the lead-in dose of cabotegravir or a pharmaceutically acceptable salt thereof is a once-daily dose of 10 mg acid equivalent and the lead-in dose of rilpivirine or a pharmaceutically acceptable salt thereof is a once-daily dose of 15 mg base equivalent.

Embodiment 60. The method of embodiment 59, wherein the lead-in doses of cabotegravir or a pharmaceutically acceptable salt thereof and rilpivirine or a pharmaceutically acceptable salt thereof are administered once daily for at least 4 weeks, in particular once daily for 4 weeks.

Embodiment 61. The method of embodiment 59 or 60, wherein the lead-in doses of cabotegravir or a pharmaceutically acceptable salt thereof are administered as two 5 mg acid equivalent dispersible tablets.

Embodiment 62. The method of any one of embodiments 59 to 61, wherein the lead-in doses of rilpivirine or a pharmaceutically acceptable salt thereof are administered as six 2.5 mg base equivalent tablets, preferably as tablets dispersed in liquid.

Embodiment 63. The method of any one of embodiments 59 to 62, wherein the lead-in doses of rilpivirine or a pharmaceutically acceptable salt thereof are provided by a pharmaceutically acceptable salt of rilpivirine.

Embodiment 64. The method of embodiment 63, wherein the pharmaceutically acceptable salt of rilpivirine is rilpivirine hydrochloride.

Embodiment 65. The method of any one of embodiments 26 to 28, further comprising orally administering lead-in doses of cabotegravir or a pharmaceutically acceptable salt thereof and rilpivirine or a pharmaceutically acceptable salt thereof prior to the loading dose, wherein the lead-in dose of cabotegravir or a pharmaceutically acceptable salt thereof is a once-daily dose of 10 mg acid equivalent and the lead-in dose of rilpivirine or a pharmaceutically acceptable salt thereof is a once-daily dose of 12.5 mg base equivalent.

Embodiment 66. The method of embodiment 65, wherein the lead-in doses of cabotegravir or a pharmaceutically acceptable salt thereof and rilpivirine or a pharmaceutically acceptable salt thereof are administered once daily for at least 4 weeks, in particular once daily for 4 weeks.

Embodiment 67. The method of embodiment 65 or 66, wherein the lead-in doses of cabotegravir or a pharmaceutically acceptable salt thereof are administered as two 5 mg acid equivalent dispersible tablets.

Embodiment 68. The method of any one of embodiments 65 to 67, wherein the lead-in doses of rilpivirine or a pharmaceutically acceptable salt thereof are administered as five 2.5 mg base equivalent tablets, preferably as tablets dispersed in liquid.

Embodiment 69. The method of any one of embodiments 65 to 68, wherein the lead-in doses of rilpivirine or a pharmaceutically acceptable salt thereof are provided by a pharmaceutically acceptable salt of rilpivirine.

Embodiment 70. The method of embodiment 69, wherein the pharmaceutically acceptable salt of rilpivirine is rilpivirine hydrochloride.

Embodiment 71. The method of any one of embodiments 29 to 31, further comprising orally administering lead-in doses of cabotegravir or a pharmaceutically acceptable salt thereof and rilpivirine or a pharmaceutically acceptable salt thereof prior to the loading dose, wherein the lead-in dose of cabotegravir or a pharmaceutically acceptable salt thereof is a once-daily dose of 10 mg acid equivalent and the lead-in dose of rilpivirine or a pharmaceutically acceptable salt thereof is a once-daily dose of 12.5 mg base equivalent.

Embodiment 72. The method of embodiment 71, wherein the lead-in doses of cabotegravir or a pharmaceutically acceptable salt thereof and rilpivirine or a pharmaceutically acceptable salt thereof are administered once daily for at least 4 weeks, in particular once daily for 4 weeks.

Embodiment 73. The method of embodiment 71 or 72, wherein the lead-in doses of cabotegravir or a pharmaceutically acceptable salt thereof are administered as two 5 mg acid equivalent dispersible tablets.

Embodiment 74. The method of any one of embodiments 71 to 73, wherein the lead-in doses of rilpivirine or a pharmaceutically acceptable salt thereof are administered as five 2.5 mg base equivalent tablets, preferably as tablets dispersed in liquid.

Embodiment 75. The method of any one of embodiments 71 to 74, wherein the lead-in doses of rilpivirine or a pharmaceutically acceptable salt thereof are provided by a pharmaceutically acceptable salt of rilpivirine.

Embodiment 76. The method of embodiment 75, wherein the pharmaceutically acceptable salt of rilpivirine is rilpivirine hydrochloride.

Embodiment 77. The method of any one of embodiments 47 to 76, wherein the lead-in doses of cabotegravir or a pharmaceutically acceptable salt thereof are administered in the form of one or more tablets or one or more dispersible tablets.

Embodiment 78. The method of any one of embodiments 47 to 77, wherein the lead-in doses of rilpivirine or a pharmaceutically acceptable salt thereof are administered in the form of one or more tablets or one or more tablets dispersed in liquid.

Embodiment 79. The method of any one of embodiments 47 to 78, wherein the lead-in doses of cabotegravir or a pharmaceutically acceptable salt thereof and rilpivirine or a pharmaceutically acceptable salt thereof are administered with food.

Embodiment 80. The method of any one of the preceding embodiments, wherein the human exhibits a viral load of less than or equal to 50 copies of HIV-1 RNA per mL of blood plasma prior to beginning treatment.

Embodiment 81. The method of any one of the preceding embodiments, wherein the human exhibits a viral load of less than or equal to 50 copies of HIV-1 RNA per mL of blood plasma after at least 72 weeks of treatment.

Embodiment 82. A method of treating HIV infection, the method comprising: administering to a human in need thereof a loading dose comprising (i) an intramuscular injection of cabotegravir or a pharmaceutically acceptable salt thereof and (ii) an intramuscular injection of rilpivirine or a pharmaceutically acceptable salt thereof; and thereafter administering one or more maintenance doses comprising (i) an intramuscular injection of cabotegravir or a pharmaceutically acceptable salt thereof and (ii) an intramuscular injection of rilpivirine or a pharmaceutically acceptable salt thereof;
wherein the human is 2 years of age to less than 12 years of age; and
wherein:
(a) the human weighs 35 kg to less than 40 kg; the loading dose of cabotegravir or a pharmaceutically acceptable salt thereof is administered as a 600 mg intramuscular injection, and the loading dose of rilpivirine or a pharmaceutically acceptable salt thereof is administered as a 900 mg intramuscular injection; and the one or more maintenance doses of cabotegravir or a pharmaceutically acceptable salt thereof are administered as 400 mg intramuscular injections and the one or more maintenance doses of rilpivirine or a pharmaceutically acceptable salt thereof are administered as 600 mg intramuscular injections;
(b) the human weighs 25 kg to 34.9 kg; the loading dose of cabotegravir or a pharmaceutically acceptable salt thereof is administered as a 300 mg intramuscular injection, and the loading dose of rilpivirine or a pharmaceutically acceptable salt thereof is administered as a 600 mg intramuscular injection; and the one or more maintenance doses of cabotegravir or a pharmaceutically acceptable salt thereof are administered as 200 mg intramuscular injections and the one or more maintenance doses of rilpivirine or a pharmaceutically acceptable salt thereof are administered as 450 mg intramuscular injections;
(c) the human weighs 20 kg to 24.9 kg; the loading dose cabotegravir or a pharmaceutically acceptable salt thereof is administered as a 300 mg intramuscular injection and the loading dose of rilpivirine or a pharmaceutically acceptable salt thereof is administered as a 600 mg intramuscular injection; and the one or more maintenance doses of cabotegravir or a pharmaceutically acceptable salt thereof are administered as 200 mg intramuscular injections and the one or more maintenance doses of rilpivirine or a pharmaceutically acceptable salt thereof are administered as 450 mg intramuscular injections;
(d) the human weighs 14 kg to 19.9 kg; the loading dose of cabotegravir or a pharmaceutically acceptable salt thereof is administered as a 300 mg intramuscular injection and the loading dose of rilpivirine or a pharmaceutically acceptable salt thereof is administered as a 450 mg intramuscular injection; and the one or more maintenance doses of cabotegravir or a pharmaceutically acceptable salt thereof are administered as 200 mg intramuscular injections and the one or more maintenance doses of rilpivirine or a pharmaceutically acceptable salt thereof are administered as 300 mg intramuscular injections; or
(e) the human weighs 10 kg to 13.9 kg; the loading dose of cabotegravir or a pharmaceutically acceptable salt thereof is administered as a 300 mg intramuscular injection and the loading dose of rilpivirine or a pharmaceutically acceptable salt thereof is administered as a 450 mg intramuscular injection; and the one or more maintenance doses of cabotegravir or a pharmaceutically acceptable salt thereof are administered as 200 mg intramuscular injections and the one or more maintenance doses of rilpivirine or a pharmaceutically acceptable salt thereof are administered as 300 mg intramuscular injections.

Embodiment 83. The method of embodiment 82, further comprising orally administering lead-in doses of cabotegravir or a pharmaceutically acceptable salt thereof and rilpivirine or a pharmaceutically acceptable salt thereof prior to the loading dose, wherein
(a) if the human weighs 35 kg to less than 40 kg, the lead-in dose of cabotegravir or a pharmaceutically acceptable salt thereof is a once-daily dose of 30 mg acid equivalent and the lead-in dose of rilpivirine or a pharmaceutically acceptable salt thereof is a once-daily dose of 25 mg base equivalent;
(b) if the human weighs 25 kg to 34.9 kg, the lead-in dose of cabotegravir or a pharmaceutically acceptable salt thereof is a once-daily dose of 10 mg acid equivalent and the lead-in dose of rilpivirine or a pharmaceutically acceptable salt thereof is a once-daily dose of 25 mg base equivalent;
(c) if the human weighs 20 kg to 24.9 kg, the lead-in dose of cabotegravir or a pharmaceutically acceptable salt thereof is a once-daily dose of 10 mg acid equivalent and the lead-in dose of rilpivirine or a pharmaceutically acceptable salt thereof is a once-daily dose of 15 mg base equivalent;
(d) if the human weighs 14 kg to 19.9 kg, the lead-in dose of cabotegravir or a pharmaceutically acceptable salt thereof is a once-daily dose of 10 mg acid equivalent and the lead-in dose of rilpivirine or a pharmaceutically acceptable salt thereof is a once-daily dose of 12.5 mg base equivalent; or
(e) if the human weighs 10 kg to 13.9 kg, the lead-in dose of cabotegravir or a pharmaceutically acceptable salt thereof is a once-daily dose of 10 mg acid equivalent and the lead-in dose of rilpivirine or a pharmaceutically acceptable salt thereof is a once-daily dose of 12.5 mg base equivalent.

Embodiment 84. Use of (i) cabotegravir or a pharmaceutically acceptable salt thereof and (ii) rilpivirine or a pharmaceutically acceptable salt thereof in the manufacture of a medicament for use in treatment of HIV infection in a human, wherein the human is 2 years of age to less than 12 years of age, and the medicament is formulated for regular intramuscular injections.

Embodiment 85. Cabotegravir or a pharmaceutically acceptable salt thereof and rilpivirine or a pharmaceutically acceptable salt thereof for use in treatment of HIV infection in a human, wherein the human is 2 years of age to less than 12 years of age, and the cabotegravir or a pharmaceutically acceptable salt thereof and rilpivirine or a pharmaceutically acceptable salt thereof are formulated for regular intramuscular injections.

Embodiment 86. Cabotegravir or a pharmaceutically acceptable salt thereof indicated in combination with rilpivirine or a pharmaceutically acceptable salt thereof for use in treatment of HIV infection in a human, wherein the human is 2 years of age to less than 12 years of age, and the cabotegravir or a pharmaceutically acceptable salt thereof is formulated for regular intramuscular injections.

Embodiment 87. Rilpivirine or a pharmaceutically acceptable salt thereof indicated in combination with cabotegravir or a pharmaceutically acceptable salt thereof for use in treatment of HIV infection in a human, wherein the human is 2 years of age to less than 12 years of age, and the rilpivirine or a pharmaceutically acceptable salt thereof is formulated for regular intramuscular injections.

It is understood that any and all embodiments of the present disclosure may be taken in combination with any other embodiment or embodiments to describe additional more preferred embodiments. It is also to be understood that each individual element of the preferred embodiments is its own independent preferred embodiment. Furthermore, any element of an embodiment is meant to be combined with any and all other elements from any embodiment to describe an additional embodiment.

### EXAMPLES

### Example 1: Phase I/II Trial of CAB LA and RPV LA in Children

### Study Design.

A Phase I/II, multicenter, open-label, non-comparative study will evaluate the safety, tolerability, acceptability, and PK of oral CAB and oral RPV followed by long-acting injectable CAB (CAB LA) and long-acting injectable RPV (RPV LA) for the weight-band dosing in virologically suppressed children living with HIV-1 aged two to less than 12 years. The study will also assess the long-acting injectable regimen with and without an oral lead-in period in the same study population. CAB LA is formulated as a suspension containing 200 mg/ml of cabotegravir free acid for administration by IM injection. RPV LA is formulated as a suspension containing 300 mg/mL of RPV free base for administration by IM injection.

This study will be conducted among up to 90 children weighing ≥10 kgs and <40 kgs, who are virologically suppressed on stable ART. Approximately 90 parents/caregivers of child participants will also enroll to complete tolerability and acceptability assessments. In-depth qualitative interviews with a subset of enrolled parents/caregivers will also be conducted. Unless otherwise noted, the term ‘participant’ will refer to the children enrolled in the study.

The study will include two cohorts.

In Cohort 1, participants will be enrolled across five weights bands as follows: Weight Band 1 (35-<40 kg), Weight Band 2 (25-34.9 kg), Weight Band 3 (20-24.9 kg), Weight Band 4 (14-19.9 kg), and Weight Band 5 (10-13.9 kg).

Once enrolled into Cohort 1, participants in each weight band will move through two steps of study participation. In Step 1 (i.e., at study entry), participants will switch from their pre-study ART regimen to daily oral formulations of CAB and oral formulations of RPV for at least four weeks and up to a maximum of six weeks. Participants who meet the eligibility criteria based on Week 4a visit will transition to Step 2 and receive injectable CAB LA+injectable RPV LA through Week 72. Participants who prematurely and permanently discontinue oral CAB+RPV or do not meet eligibility criteria for the injection phase will exit the study 28 days after their last oral study product dosing. In Step 2, participants will receive injectable CAB LA+RPV LA. Two IM injections, a single injection of CAB LA and a single injection of RPV LA, will be administered at the Week 4b (Step 2 Entry) visit, at the Week 8 visit, and then continuing every four weeks or every eight weeks thereafter, depending on the recommendation of the Week 12 interim analyses, with the last injections administered at the Week 72 visit. In both steps, participants will follow the dosing regimens outlined in the Product Regimen section below.

Cohort 2 will consist of two groups, Cohort 2a and Cohort 2b, which will allow participants to choose between a regimen that include an oral-lead in and one that does not. Participants enrolled in Cohort 2a will progress through two steps of study participation, as described above for Cohort 1. Cohort 2a participants will receive oral CAB+oral RPV (Step 1) through the Week 4b visit, followed by intramuscular injection doses of CAB LA and injectable doses of RPV LA from Week 4b (Step 2 entry) through Week 48, based upon the relevant weight band dosing regimens recommended at the time. Participants enrolled to Cohort 2b will skip the oral lead-in phase and receive both CAB LA and RPV LA upon entry into the study and continuing through Week 44, based upon the relevant weight-band dosing regimens recommended at the time.

The decision to join Cohort 2a or Cohort 2b will be made by the potential study participant and/or the parent/legal guardian in consultation with the investigator of record or designee and other healthcare providers, as applicable. This choice will be made as part of the informed consent or assent process and must be confirmed prior to study entry; participants may not change their Cohort 2a or Cohort 2b group selection after enrollment.

### Product Regimen.

At Entry, all participants will discontinue their pre-study cART regimen and will be assigned to a dosing regimen based on their weight at entry to the applicable cohort. Weight-based dose adjustments will made according to guidance provided in the Dose Adjustments section below. Dosing regimens may be modified following the Cohort 1 interim analyses or based on experience in the study, in which case, the new regimen will be specified per the Dosing Regimen Modifications section below.

Cohort 1 participants will receive oral CAB and oral RPV followed by intramuscular CAB LA and intramuscular RPV LA as shown in Table 1 (for oral dosing) and Table 2 (for LA injections).

Cohort 2a participants will receive oral CAB and oral RPV followed by intramuscular CAB LA and intramuscular RPV LA. Cohort 2b participants will receive intramuscular CAB LA and intramuscular RPV LA only.

TABLE 1
Cohort 1: Initial Oral Lead-In Dosing Regimen

| | Daily Oral Dose |
|---|---|
| Weight Band | (through the Week 4b visit) |
| Weight Band 1 | 30 mg CAB + 25 mg RPV |
| 35-<40 kg | |
| Weight Band 2 | 10 mg CAB + 25 mg RPV |
| 25-34.9 kg | Administered as two 5 mg CAB DT + |
| | one 25 mg RPV tablet |
| Weight Band 3 | 10 mg CAB + 15 mg RPV |
| 20-24.9 kg | Administered as two 5 mg CAB DT + |
| | six 2.5 mg RPV tablets |
| Weight Band 4 | 10 mg CAB + 12.5 mg RPV |
| 14-19.9 kg | Administered as two 5 mg CAB DT + |
| | five 2.5 mg RPV tablets |
| Weight Band 5 | 10 mg CAB + 12.5 mg RPV |
| 10-13.9 kg | Administered as two 5 mg CAB DT + |
| | five 2.5 mg RPV tablets |

TABLE 2
Cohort 1: Initial (Q4W) IM Injection Dosing Regimen

| Weight Band | Q4W Long-acting IM Dose Regimen |
|---|---|
| Weight Band 1 | Initial injections (Wk 4b): |
| 35-<40 kg | 600 mg CAB LA + 900 mg RPV LA |
| | Subsequent injections (following |
| | Wk 4b), every four weeks: |
| | 400 mg CAB LA + 600 mg RPV LA |
| Weight Band 2 | Initial injections (Wk 4b): |
| 25-34.9 kg | 300 mg CAB LA + 600 mg RPV LA |
| | Subsequent injections (following |
| | Wk 4b), every four weeks: |
| | 200 mg CAB LA + 450 mg RPV LA |
| Weight Band 3 | Initial injections (Wk 4b): |
| 20-24.9 kg | 300 mg CAB LA + 600 mg RPV LA |
| | Subsequent injections (following |
| | Wk 4b), every four weeks: |
| | 200 mg CAB LA + 450 mg RPV LA |
| Weight Band 4 | Initial injections (Wk 4b): |
| 14-19.9 kg | 300 mg CAB LA + 450 mg RPV LA |
| | Subsequent injections (following |
| | Wk 4b), every four weeks: |
| | 200 mg CAB LA + 300 mg RPV LA |
| Weight Band 5 | Initial injections (Wk 4b): |
| 10-13.9 kg | 300 mg CAB LA + 450 mg RPV LA |
| | Subsequent injections (following |
| | Wk 4b), every four weeks: |
| | 200 mg CAB LA + 300 mg RPV LA |

Based on modeling and analysis from prior data, initial doses were determined for each weight band, and the oral and long-acting regimens with the proposed initial doses in Tables 1 and 2 are calculated to achieve plasma concentrations within an acceptable range of those observed clinically in adolescents and adults.

### Dose Adjustments.

Participant weight at the Entry visit will establish the participant's weight band and the assigned oral dosing for the full duration of the oral lead-in phase. Oral dosing will not be adjusted due to weight band changes (increases or decreases in weight) which may occur during the oral lead-in phase.

At each injection visit, participant weight will be assessed prior to administering study product to determine the appropriate weight band and injectable dosing regimen to be administered at that visit. Participants who increase in weight bands will begin the dosing regimen corresponding to their new applicable weight band. However, decreases in weight band will not result in a dose change; participants will remain on the dose regimen of the largest weight band achieved.

### Dosing Regimen Modifications.

Dosing regimen modifications for each weight band may occur as needed following the Cohort 1 interim analyses and/or based on ongoing reviews of safety, PK, viral load, tolerability, and all other relevant data within the study or from other ongoing studies of the study drugs in pediatric populations. Dose modifications, and the initial doses for Cohort 2 as noted above, will be selected from the Tables 3 to 8. Currently enrolled and newly enrolling participants will be assigned to the updated dosing regimens.

In the event of an injection regimen modification from Q4W to Q8W dosing, enrolled participants still in the oral lead-in phase can receive injections on the Q8W schedule. Enrolled participants who have initiated injections on a Q4W injection schedule and have completed the Week 12 visit will continue to receive injections on a Q4W schedule through the Week 24 (Cohort 1 and Cohort 2a) or Week 20 (Cohort 2b) visit.

Regardless of dosing regimen modification, it is contemplated that each dosing regimen will include, where applicable, daily oral doses of cabotegravir and rilpivirine shown in Tables 3 and 4, injectable CAB LA+RPV LA doses shown in Tables 5, 6, 7, and 8, and an injection intervals shown in Table 9.

TABLE 3
CAB Oral Dose

| | | |
|---|---|---|
| III.A | 30 mg | Administered as one 30 mg CAB tablet |
| III.B | 25 mg | Administered as five 5 mg CAB DT in a split dose |
| III.C | 20 mg | Administered as four 5 mg CAB DT |
| III.D | 15 mg | Administered as three 5 mg CAB DT |
| III.E | 10 mg | Administered as two 5 mg CAB DT |
| III.F | 5 mg | Administered as one 5 mg CAB DT |

TABLE 4
RPV Oral Dose

| | | |
|---|---|---|
| IV.A | 25 mg | Administered as one 25 mg RPV tablet |
| IV.B | 20 mg | Administered as eight 2.5 mg RPV tablets |
| IV.C | 15 mg | Administered as six 2.5 mg RPV tablets |
| IV.D | 12.5 mg | Administered as five 2.5 mg RPV tablets |
| IV.E | 10 mg | Administered as four 2.5 mg RPV tablets |
| IV.F | 7.5 mg | Administered as three 2.5 mg RPV tablets |
| IV.G | 5 mg | Administered as two 2.5 mg RPV tablets |
| IV.H | 2.5 mg | Administered as one 2.5 mg RPV tablets |

TABLE 5
CAB LA First Injection Dose

| | |
|---|---|
| V.A | 600 mg |
| V.B | 400 mg |
| V.C | 300 mg |
| V.D | 200 mg |
| V.E | 100 mg |

TABLE 6
CAB LA Subsequent Injections

| | |
|---|---|
| VI.A | 600 mg |
| VI.B | 400 mg |
| VI.C | 300 mg |
| VI.D | 200 mg |
| VI.E | 100 mg |

TABLE 7
RPV LA First Injection Dose

| | |
|---|---|
| VII.A | 900 mg |
| VII.B | 750 mg |
| VII.C | 600 mg |
| VII.D | 450 mg |
| VII.E | 300 mg |
| VII.F | 150 mg |

TABLE 8
RPV LA Subsequent Injection Dose

| | |
|---|---|
| VIII.A | 900 mg |
| VIII.B | 750 mg |
| VIII.C | 600 mg |
| VIII.D | 450 mg |
| VIII.E | 300 mg |
| VIII.F | 150 mg |

TABLE 9
Injection Interval

| | |
|---|---|
| IX.A | Q. 4 weeks |
| IX.B | Doses 1 and 2, 4 weeks apart, then Q. 8 weeks |

### Oral Bridging for Participants Receiving LA Injectables

In cases, participants who will miss a scheduled injection may receive daily oral CAB+RPV as a bridging strategy. The oral study product weight band dosing regimen will be assigned according to the participant's weight obtained at the most recent study visit (whether a regularly scheduled visit or an interim visit).

Participants are to ideally begin the oral bridging regimen on the same target visit date (or within the same target visit window) as that of the missed injection visit. The last dose of the short-term oral bridging regimen should be taken on the same day as and prior to resuming injectable study product. Step 2 participants may be required to have interim injection visits upon resuming the study product injections to appropriately reinitiate the dosing regimen or to realign to the original injection visit dosing schedule.

What is claimed is:

1. A method of treating HIV infection, the method comprising:
administering to a human in need thereof a loading dose comprising (i) an intramuscular injection of cabotegravir or a pharmaceutically acceptable salt thereof and (ii) an intramuscular injection of rilpivirine or a pharmaceutically acceptable salt thereof; and
thereafter administering one or more maintenance doses comprising (i) an intramuscular injection of cabotegravir or a pharmaceutically acceptable salt thereof and (ii) an intramuscular injection of rilpivirine or a pharmaceutically acceptable salt thereof;
wherein the human is 2 years of age to less than 12 years of age.

2. The method of claim 1, wherein the human weighs 10 kg to less than 40 kg, preferably 10 kg to 34.9 kg.

3. The method of claim 1, wherein the cabotegravir or a pharmaceutically acceptable salt thereof is administered as a 50 mg to 600 mg intramuscular injection, preferably a 100 mg to 600 mg intramuscular injection, a 150 mg to 600 mg intramuscular injection, a 200 mg to 600 mg intramuscular injection, a 300 mg to 600 mg intramuscular injection, or a 200 mg to 400 mg intramuscular injection, and the rilpivirine or a pharmaceutically acceptable salt thereof is administered as a 50 mg to 900 mg intramuscular injection, preferably a 100 mg to 900 mg intramuscular injection, a 150 mg to 900 mg intramuscular injection, a 200 mg to 900 mg intramuscular injection, a 250 mg to 900 mg intramuscular injection, a 300 mg to 900 mg intramuscular injection, a 450 mg to 900 mg intramuscular injection, or a 300 mg to 600 mg intramuscular injection.

4. The method of claim 1, wherein the one or more maintenance doses are administered once every 4 weeks ±7 days or less frequently.

5. The method of claim 1, wherein the injections of cabotegravir or a pharmaceutically acceptable salt thereof and rilpivirine or a pharmaceutically acceptable salt thereof are in the form of long-acting formulations.

6. The method of claim 1, wherein the injections of cabotegravir or a pharmaceutically acceptable salt thereof are formulated as suspensions and the cabotegravir or a pharmaceutically acceptable salt thereof is in free acid/base form; and the injections of rilpivirine or a pharmaceutically acceptable salt thereof are formulated as suspensions and the rilpivirine or a pharmaceutically acceptable salt thereof is in free base form.

7. The method of claim 1, further comprising orally administering lead-in doses of cabotegravir or a pharmaceutically acceptable salt thereof and rilpivirine or a pharmaceutically acceptable salt thereof prior to the loading dose.

8. The method of claim 7, wherein the lead-in doses of cabotegravir or a pharmaceutically acceptable salt thereof are daily doses of 5 mg to 30 mg acid equivalent, preferably 10 mg to 30 mg acid equivalent, and the lead-in doses of rilpivirine or a pharmaceutically acceptable salt thereof are daily doses of 2.5 mg to 25 mg base equivalent, preferably 12.5 mg to 25 mg base equivalent.

9. The method of claim 7 or 8, wherein the lead-in doses of cabotegravir or a pharmaceutically acceptable salt thereof are administered in the form of one or more tablets or one or more dispersible tablets, and the lead-in doses of rilpivirine or a pharmaceutically acceptable salt thereof are administered in the form of one or more tablets or one or more tablets dispersed in liquid.

10. The method of claim 7, wherein the lead-in doses of rilpivirine or a pharmaceutically acceptable salt thereof are provided by a pharmaceutically acceptable salt of rilpivirine, preferably wherein the pharmaceutically acceptable salt of rilpivirine is rilpivirine hydrochloride.

11. A method of treating HIV infection, the method comprising:
administering to a human in need thereof a loading dose comprising (i) an intramuscular injection of cabotegravir or a pharmaceutically acceptable salt thereof and (ii) an intramuscular injection of rilpivirine or a pharmaceutically acceptable salt thereof; and
thereafter administering one or more maintenance doses comprising (i) an intramuscular injection of cabotegravir or a pharmaceutically acceptable salt thereof and (ii) an intramuscular injection of rilpivirine or a pharmaceutically acceptable salt thereof;
wherein the human is 2 years of age to less than 12 years of age; and
wherein:
(a) the human weighs 35 kg to less than 40 kg; the loading dose of cabotegravir or a pharmaceutically acceptable salt thereof is administered as a 600 mg intramuscular injection, and the loading dose of rilpivirine or a pharmaceutically acceptable salt thereof is administered as a 900 mg intramuscular injection; and the one or more maintenance doses of cabotegravir or a pharmaceutically acceptable salt thereof are administered as 400 mg intramuscular injections and the one or more maintenance doses of rilpivirine or a pharmaceutically acceptable salt thereof are administered as 600 mg intramuscular injections;
(b) the human weighs 25 kg to 34.9 kg; the loading dose of cabotegravir or a pharmaceutically acceptable salt thereof is administered as a 300 mg intramuscular injection, and the loading dose of rilpivirine or a pharmaceutically acceptable salt thereof is administered as a 600 mg intramuscular injection; and the one or more maintenance doses of cabotegravir or a pharmaceutically acceptable salt thereof are administered as 200 mg intramuscular injections and the one or more maintenance doses of rilpivirine or a pharmaceutically acceptable salt thereof are administered as 450 mg intramuscular injections;
(c) the human weighs 20 kg to 24.9 kg; the loading dose cabotegravir or a pharmaceutically acceptable salt thereof is administered as a 300 mg intramuscular injection and the loading dose of rilpivirine or a pharmaceutically acceptable salt thereof is administered as a 600 mg intramuscular injection; and the one or more maintenance doses of cabotegravir or a pharmaceutically acceptable salt thereof are administered as 200 mg intramuscular injections and the one or more maintenance doses of rilpivirine or a pharmaceutically acceptable salt thereof are administered as 450 mg intramuscular injections;
(d) the human weighs 14 kg to 19.9 kg; the loading dose of cabotegravir or a pharmaceutically acceptable salt thereof is administered as a 300 mg intramuscular injection and the loading dose of rilpivirine or a pharmaceutically acceptable salt thereof is administered as a 450 mg intramuscular injection; and the one or more maintenance doses of cabotegravir or a pharmaceutically acceptable salt thereof are administered as 200 mg intramuscular injections and the one or more maintenance doses of rilpivirine or a pharmaceutically acceptable salt thereof are administered as 300 mg intramuscular injections; or
(e) the human weighs 10 kg to 13.9 kg; the loading dose of cabotegravir or a pharmaceutically acceptable salt thereof is administered as a 300 mg intramuscular injection and the loading dose of rilpivirine or a pharmaceutically acceptable salt thereof is administered as a 450 mg intramuscular injection; and the one or more maintenance doses of cabotegravir or a pharmaceutically acceptable salt thereof are administered as 200 mg intramuscular injections and the one or more maintenance doses of rilpivirine or a pharmaceutically acceptable salt thereof are administered as 300 mg intramuscular injections.

12. The method of claim 11, further comprising orally administering lead-in doses of cabotegravir or a pharmaceutically acceptable salt thereof and rilpivirine or a pharmaceutically acceptable salt thereof prior to the loading dose, wherein
(a) if the human weighs 35 kg to less than 40 kg, the lead-in dose of cabotegravir or a pharmaceutically acceptable salt thereof is a once-daily dose of 30 mg acid equivalent and the lead-in dose of rilpivirine or a pharmaceutically acceptable salt thereof is a once-daily dose of 25 mg base equivalent;
(b) if the human weighs 25 kg to 34.9 kg, the lead-in dose of cabotegravir or a pharmaceutically acceptable salt thereof is a once-daily dose of 10 mg acid equivalent and the lead-in dose of rilpivirine or a pharmaceutically acceptable salt thereof is a once-daily dose of 25 mg base equivalent;
(c) if the human weighs 20 kg to 24.9 kg, the lead-in dose of cabotegravir or a pharmaceutically acceptable salt thereof is a once-daily dose of 10 mg acid equivalent and the lead-in dose of rilpivirine or a pharmaceutically acceptable salt thereof is a once-daily dose of 15 mg base equivalent;
(d) if the human weighs 14 kg to 19.9 kg, the lead-in dose of cabotegravir or a pharmaceutically acceptable salt thereof is a once-daily dose of 10 mg acid equivalent and the lead-in dose of rilpivirine or a pharmaceutically acceptable salt thereof is a once-daily dose of 12.5 mg base equivalent; or
(e) if the human weighs 10 kg to 13.9 kg, the lead-in dose of cabotegravir or a pharmaceutically acceptable salt thereof is a once-daily dose of 10 mg acid equivalent and the lead-in dose of rilpivirine or a pharmaceutically acceptable salt thereof is a once-daily dose of 12.5 mg base equivalent.

13. (canceled)

14. (canceled)

15. (canceled)

16. (canceled)
